SEQUENCE LISTING (52 sequences)

SEQ ID NO: 1 - protein, 15 aa, Artificial Sequence
  Description of Artificial Sequence Synthetic peptide
LVVLHSELHKLKSEL

SEQ ID NO: 2 - protein, 15 aa, Artificial Sequence
  Description of Artificial Sequence Synthetic peptide
LVVLHSHLEKLKSEL

SEQ ID NO: 3 - protein, 26 aa, Artificial Sequence
  Description of Artificial Sequence Synthetic peptide
LKVELEKLKSELVVLHSELHKLKSEL

SEQ ID NO: 4 - protein, 26 aa, Artificial Sequence
  Description of Artificial Sequence Synthetic peptide
LKVELEKLKSELVVLHSHLEKLKSEL

SEQ ID NO: 5 - protein, 26 aa, Artificial Sequence
  Description of Artificial Sequence Synthetic peptide
LKVELKELKKELVVLKSELKELKKEL

SEQ ID NO: 6 - protein, 11 aa, Artificial Sequence
  Description of Artificial Sequence Synthetic peptide
QQKFQFQFEQQ

SEQ ID NO: 7 - protein, 11 aa, Artificial Sequence
  Description of Artificial Sequence Synthetic peptide
QQKFQFQFHQQ

SEQ ID NO: 8 - protein, 8 aa, Artificial Sequence
  Description of Artificial Sequence Synthetic peptide
FKFEFKFE

SEQ ID NO: 9 - protein, 7 aa, Artificial Sequence
  Description of Artificial Sequence Synthetic peptide
KFQFQFE

SEQ ID NO: 10 - protein, 11 aa, Artificial Sequence
  Description of Artificial Sequence Synthetic peptide
QQRFQFQFEQQ

SEQ ID NO: 11 - protein, 11 aa, Artificial Sequence
  Description of Artificial Sequence Synthetic peptide
QQRFQWQFEQQ

SEQ ID NO: 12 - protein, 16 aa, Artificial Sequence
  Description of Artificial Sequence Synthetic peptide
FEFEFKFKFEFEFKFK

SEQ ID NO: 13 - protein, 11 aa, Artificial Sequence
  Description of Artificial Sequence Synthetic peptide
QQRFEWEFEQQ

SEQ ID NO: 14 - protein, 11 aa, Artificial Sequence
  Description of Artificial Sequence Synthetic peptide
  MOD_RES (3)..(3): Ornithine
  MOD_RES (5)..(5): Ornithine
  MOD_RES (7)..(7): Ornithine
QQXFXWXFQQQ

SEQ ID NO: 15 - protein, 12 aa, Artificial Sequence
  Description of Artificial Sequence Synthetic peptide
FKFEFKFEFKFE

SEQ ID NO: 16 - protein, 12 aa, Artificial Sequence
  Description of Artificial Sequence Synthetic peptide
FKFQFKFQFKFQ

SEQ ID NO: 17 - protein, 16 aa, Artificial Sequence
  Description of Artificial Sequence Synthetic peptide
AEAKAEAKAEAKAEAK

SEQ ID NO: 18 - protein, 16 aa, Artificial Sequence
  Description of Artificial Sequence Synthetic peptide
AEAEAKAKAEAEAKAK

SEQ ID NO: 19 - protein, 16 aa, Artificial Sequence
  Description of Artificial Sequence Synthetic peptide
AEAEAEAEAKAKAKAK

SEQ ID NO: 20 - protein, 16 aa, Artificial Sequence
  Description of Artificial Sequence Synthetic peptide
RADARADARADARADA

SEQ ID NO: 21 - protein, 16 aa, Artificial Sequence
  Description of Artificial Sequence Synthetic peptide
RARADADARARADADA

SEQ ID NO: 22 - protein, 33 aa, Artificial Sequence
  Description of Artificial Sequence Synthetic polypeptide
SGRGYBLGGQGAGAAAAAGGAGQGGYGGLGSQG

SEQ ID NO: 23 - protein, 10 aa, Artificial Sequence
  Description of Artificial Sequence Synthetic peptide
  MOD_RES (4)..(4): Val, Ala, Ser, or Pro
  MOD_RES (6)..(6): Val, Ala, Ser, or Pro
  MOD_RES (8)..(8): Val, Ala, Ser, or Pro
  MOD_RES (10)..(10): Val, Ala, Ser, or Pro
EWEXEXEXEX

SEQ ID NO: 24 - protein, 10 aa, Artificial Sequence
  Description of Artificial Sequence Synthetic peptide
  MOD_RES (3)..(3): Val, Ala, Ser, or Pro
  MOD_RES (5)..(5): Val, Ala, Ser, or Pro
  MOD_RES (7)..(7): Val, Ala, Ser, or Pro
  MOD_RES (9)..(9): Val, Ala, Ser, or Pro
WKXKXKXKXK

SEQ ID NO: 25 - protein, 15 aa, Artificial Sequence
  Description of Artificial Sequence Synthetic peptide
KWKVKVKVKVKVKVK

SEQ ID NO: 26 - protein, 16 aa, Artificial Sequence
  Description of Artificial Sequence Synthetic peptide
LLLLKKKKKKKKLLLL

SEQ ID NO: 27 - protein, 21 aa, Artificial Sequence
  Description of Artificial Sequence Synthetic peptide
VKVKVKVKVDPPTKVKVKVKV

SEQ ID NO: 28 - protein, 21 aa, Artificial Sequence
  Description of Artificial Sequence Synthetic peptide
VKVKVKVKVDPPTKVKTKVKV

SEQ ID NO: 29 - protein, 21 aa, Artificial Sequence
  Description of Artificial Sequence Synthetic peptide
KVKVKVKVKDPPSVKVKVKVK

SEQ ID NO: 30 - protein, 21 aa, Artificial Sequence
  Description of Artificial Sequence Synthetic peptide
VKVKVKVKVDPPSKVKVKVKV

SEQ ID NO: 31 - protein, 21 aa, Artificial Sequence
  Description of Artificial Sequence Synthetic peptide
VKVKVKTKVDPPTKVKTKVKV

SEQ ID NO: 32 - protein, 16 aa, Artificial Sequence
  Description of Artificial Sequence Synthetic peptide
KKSLSLSLSLSLSLKK

SEQ ID NO: 33 - protein, 10 aa, Artificial Sequence
  Description of Artificial Sequence Synthetic peptide
YTIAALLSPY

SEQ ID NO: 34 - protein, 19 aa, Artificial Sequence
  Description of Artificial Sequence Synthetic peptide
  MOD_RES (3)..(3): Any amino acid
LPXTGGGGGGGGGGGGGGG

SEQ ID NO: 35 - protein, 15 aa, Artificial Sequence
  Description of Artificial Sequence Synthetic peptide
GGGGGGGGGGGGGGG

SEQ ID NO: 36 - protein, 6 aa, Artificial Sequence
  Description of Artificial Sequence Synthetic peptide
LPETGG

SEQ ID NO: 37 - protein, 6 aa, Artificial Sequence
  Description of Artificial Sequence Synthetic peptide
LPSTGG

SEQ ID NO: 38 - protein, 19 aa, Artificial Sequence
  Description of Artificial Sequence Synthetic peptide
  MOD_RES (3)..(3): Any amino acid
LPXTGGGGGGGGGGGGGGG

SEQ ID NO: 39 - protein, 16 aa, Artificial Sequence
  Description of Artificial Sequence Synthetic peptide
CSGSGQQKPQPQPEQQ

SEQ ID NO: 40 - protein, 4 aa, Unknown
  Description of Unknown cleavage site sequence
REKR

SEQ ID NO: 41 - protein, 9 aa, Unknown
  Description of Unknown cleavage site sequence
RRVVEREKR

SEQ ID NO: 42 - protein, 9 aa, Unknown
  Description of Unknown cleavage site sequence
ERVVEREKE

SEQ ID NO: 43 - protein, 4 aa, Unknown
  Description of Unknown cleavage site sequence
SEKS

SEQ ID NO: 44 - protein, 79 aa, Unknown
  Description of Unknown envelope sequence
MRVMGIQRNYPQWWIWSMLGFWMLMICNGMWVTVYYGVPVWKEAKTTLFCASDAKAYEKEVHNVWATHACVPTDPNPQE

SEQ ID NO: 45 - protein, 24 aa, Homo sapiens
MPMGSLQPLATLYLLGMLVASVLA

SEQ ID NO: 46 - protein, 28 aa, Artificial Sequence
  Description of Artificial Sequence Synthetic peptide
  MOD_RES (1)..(1): D-Alanine
  MOD_RES (3)..(3): Cyclohexylalanine
  MOD_RES (13)..(13): D-Alanine
XKXVAAWTLKAAXSGSGQQKFQFQFEQQ

SEQ ID NO: 47 - protein, 5 aa, Unknown
  Description of Unknown recognition motif sequence
  MOD_RES (3)..(3): Any amino acid
LPXTG

SEQ ID NO: 48 - protein, 5 aa, Artificial Sequence
  Description of Artificial Sequence Synthetic peptide
LPETG

SEQ ID NO: 49 - DNA, 1982 bp, Artificial Sequence
  Description of Artificial Sequence Synthetic polynucleotide
tctagaccaccatgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatctgcctagcaccggaggatgatgaggatcc

SEQ ID NO: 50 - protein, 653 aa, Artificial Sequence
  Description of Artificial Sequence Synthetic polypeptide
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPSTGG

SEQ ID NO: 51 - protein, 27 aa, Artificial Sequence
  Description of Artificial Sequence Synthetic peptide
  MOD_RES (1)..(1): D-Alanine
  MOD_RES (3)..(3): Cyclohexylalanine
  MOD_RES (12)..(12): D-Alanine
XKXVAAWTLKAXSGSGQQKFQFQFEQQ

SEQ ID NO: 52 - protein, 5 aa, Unknown
  Description of Unknown recognition motif sequence
  MOD_RES (4)..(4): Any amino acid
LPTXG

(19) United States
(12) Patent Application Publication
(10) Pub. No.: US 2022/0040290 A1
(43) Pub. Date: Feb. 10, 2022

(54) COMPOSITIONS COMPRISING SUPRAMOLECULAR NANOFIBER HIV ENVELOPES AND METHODS FOR THEIR USE

(71) Applicant: Duke University, Durham, NC (US)

(72) Inventors: Sallie PERMAR, Durham, NC (US); Joel COLLIER, Durham, NC (US); Kevin O. SAUNDERS, Durham, NC (US); Chelsea FRIES, Durham, NC (US); Fouda Amou'ou Genevieve GINY, Durham, NC (US)

(21) Appl. No.: 17/279,033

(22) PCT Filed: Sep. 24, 2019
(86) PCT No.: PCT/US2019/052637

§ 371 Date: Mar. 23, 2021

Related U.S. Application Data
(60) Provisional application No. 62/735,781, filed on Sep. 24, 2018.

(51) Int. Cl. A61K 39/21; A61P 31/18

(52) U.S. Cl. CPC A61K 39/21; A61K 2039/6031; A61P 31/18

(57) ABSTRACT

The technology provides immunogenic compositions comprising HIV-1 envelopes in supramolecular nanofiber complexes, which may also comprise a T-cell helper epitopes, and methods of using these compositions for induction of immune responses.

This application claims the benefit and priority of U.S. Application Ser. No. 62/735,781 filed Sep. 24, 2018 which content is herein incorporated by reference in its entirety.

This invention was made with government support under Center for HIV/AIDS Vaccine Immunology-Immunogen Design grant UM1-AI100645 from the NIH, NIAID, Division of AIDS and government support from the Duke University Center for AIDS Research (CFAR), an NIH funded program (5P30 AI064518) and under NTH grant 1R01AI145016. The government has certain rights in the invention.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and method for induction of immune response, for example cross-reactive (broadly) neutralizing Ab induction.

In certain embodiments the invention provides immunogenic compositions comprising HIV-1 envelopes in supramolecular nanofiber complex. In certain embodiments, the supramolecular nanofiber complex also comprises a T-cell helper epitope, for example but not limited to PADRE peptide. Nanofiber complex technology is disclosed in U.S. Pat. No. 9,200,082, which contents are herein incorporated by reference in their entirety. Self-assembled, multi-component matrices using a short fibrillizing peptide, Q11 is disclosed in U.S. Pat. No. 9,849,174, which contents are herein incorporated by reference in their entirety.

In certain embodiments the envelope is any of the forms of HIV-1 envelope. In certain embodiments the envelope is gp120, gp140, gp145 (i.e. with a transmembrane domain), or gp150. In certain embodiments, gp140 is designed to form a stable trimer. See WO/2017/15180, e.g. Table 1, FIGS. 22-24, Example 9, and paragraphs [0501] et seq. for non-limiting examples of sequences of stable trimer designs, which contents are incorporated by reference in their entirety. In certain embodiments envelope protomers form a trimer which is not a SOSIP timer. In certain embodiments the trimer is a SOSIP based trimer wherein each protomer comprises additional modifications. In certain embodiments, envelope trimers are recombinantly produced.

In certain aspects, the invention provides methods of inducing an immune response in a subject comprising administering a composition comprising an HIV-1 envelope(s) in any of the inventive supramolecular nanofiber formulations in an amount sufficient to induce an immune response.

In certain embodiments, the method further comprises administering an adjuvant. Any suitable adjuvant could be used. In certain embodiments, the method further comprises administering any other HIV-1 immunogen, including but not limited to other HIV-1 envelopes.

In certain aspects, the invention provides an immunogenic composition comprising a nanofiber complex composition, wherein the composition comprises a β-sheet nanofiber structure comprising a plurality of β-sheet peptides, and a compound attached via a linker to at least one of the β-sheet peptides, and wherein the compound is an HIV-1 envelope such as gp120, gp140, or a stabilized HIV-1 trimer. In certain non-limiting embodiments, the β-sheet peptide is Q11. In certain embodiments, the compound is linked to at least one of the β-sheet peptides, wherein the linker is any suitable linker. In certain embodiments the compound is attached via site-specific conjugation. In a non-limiting embodiment, the site specific conjugation is carried out via a sortase mediated reaction.

In certain aspects, the invention provides an immunogenic composition comprising a nanofiber complex composition, wherein the composition comprises a β-sheet nanofiber structure comprising a plurality of β-sheet peptides, and a compound attached to at least one of the β-sheet peptides, and wherein the compound is an HIV-1 envelope such as gp120, gp140, or a stabilized trimer. The compound could be attached to the nanofiber, including but not limited to a Q11 nanofiber via any suitable linker or chemistry. In certain embodiments the invention provides that multiple envelopes are attached to the nanofiber. The envelopes could be the same envelope, or different envelopes. The envelopes could be monomers or multimerized. In a non-limiting embodiment, the conjugation of the compound to the nanofiber is carried out via a sortase mediated reaction.

In non-limiting embodiments, the HIV-envelope is linked to at least one of the β-sheet peptides. Any suitable linker could be used to attach the envelope to the beta-sheet peptide.

Non-limiting embodiments are shown in FIGS. 26, 28, and 31. In some embodiments, the envelope has a linker used in the sortase reaction, wherein the linker is LPXTG₁₅-beta tail. The sortase enzyme reaction is known in the art.

In certain embodiments, the compound is gp120 HIV-1 envelope 1086.C.

In certain embodiments, the compound is HIV-1 envelope trimer, wherein in certain non-limiting embodiments, the HIV-1 envelops trimer is CH505 T/F. See instant Example 1 and FIGS. 18A-B; see also FIG. 24 in WO/2017/15180.

In certain embodiments, the plurality of β-sheet peptides comprises a plurality of self-assembling peptides.

In certain embodiments, the β-sheet peptide is Q11.

In certain embodiments, the nanofiber complex composition comprises a T-cell epitope peptide. Any suitable T cell epitope could be used. In certain embodiments, the nanofiber complex composition comprises the PADRE peptide.

In certain embodiments, the composition further comprises an adjuvant.

In certain aspects, the invention provides a method of inducing an immune response in a subject, comprising administering to the subject any one of the inventive compositions of the invention.

In certain embodiments, the method is further comprising administering an adjuvant.

In certain aspects, the invention provides an immunogenic composition comprising a nanofiber complex composition, wherein the composition comprises a β-sheet nanofiber structure comprising
a) a plurality of non-β-sheet peptide tags that undergo a transition from a non-β-sheet structure to a β-sheet structure in the presence of β-sheet peptides, wherein a non-β-sheet peptide tag is attached to a compound; wherein the compound is an HIV-1 envelope such as gp120, gp140, or a stabilized trimer, and
b) a plurality of β-sheet peptides, wherein in certain embodiments, the β-sheet peptide is Q11.

In certain embodiments, the structure comprises at least two different compounds.

In certain embodiments, the non-β-sheet peptides tags are α-helical peptides.

In certain embodiments, non-β-sheet peptides tags comprise one or more alpha helical motifs having a sequence of a b c d e f g, with a and d being non-polar amino acids and e and g being charged amino acids.

In certain embodiments, a and/or d is Ala (A), Leu (L), Ile (I), Val (V) or a conservative derivative thereof in one or more of the alpha helical motifs.

In certain embodiments, a and/or d is Leu (L) in one or more of the alpha helical motifs.

In certain embodiments, e and/or g is Lys (K), Arg (R), His (H), Asp (D), Glu (E) or a conservative derivative thereof in one or more of the alpha helical motifs.

In certain embodiments, one or more of b, c, and f is a hydrophobic amino acid in one or more of the alpha helical motifs.

In certain embodiments, one or more of b, c, and f in one or more of the alpha helical motifs is Val (V), Tyr (Y), Phe (F), Trp (W), Ile (I), or Thr (T).

In certain embodiments, one or more of b, c, and f is Val (V) in one or more of alpha helical motifs.

In certain embodiments, the non-β-sheet peptide tag comprises an amino acid sequence having at least 90% identity with the sequence of LVVLHSELHKLKSEL (SEQ ID NO: 1), LVVLHSHLEKLKSEL (SEQ ID NO: 2), LKVELEKLKSELVVLHSELHKLKSEL (SEQ ID NO: 3), LKVELEKLKSELVVLHSHLEKLKSEL (SEQ ID NO: 4), or LKVELKELKKELVVLKSELKELKKEL (SEQ ID NO: 5). In certain embodiments, the non-β-sheet peptide tag comprises an amino acid sequence having at least 90, 91, 92, 93, 94, 95, 96, 97, 98, 99 or 100% identity with the sequence of SEQ ID NO: 1, SEQ ID NO: 2, SEQ ID NO: 3, SEQ ID NO: 4, or SEQ ID NO: 5.

In certain embodiments, one or more of the alpha helical motifs further comprise at least two metal binding amino acids spaced by one or three amino acids.

In certain embodiments, the non-β-sheet peptide tag has 14 to 56 amino acids in length.

In certain embodiments, the compound attached to the non-β-sheet peptide tags is a HIV-1 envelope, or a combination thereof.

In certain embodiments, at least one of the non-β-sheet peptide tags attached to a compound is a fusion protein.

In certain embodiments, one or more of the non-β-sheet peptide tags are attached to the amino-terminus of a peptide.

In certain embodiments the compound attached to a non-R-sheet peptide tag is an enzyme, fluorescent protein, cell binding domain, cell adhesion domain, extracellular matrix domain, reporter protein, cytokine, antigen, signaling domain, immunomodulating protein, cross-linking protein, hormone, hapten, or a combination thereof.

In certain embodiments the β-sheet peptides comprise a plurality of self-assembling peptides.

In certain embodiments the β-sheet peptide has 2 to 40 amino acids in length.

In certain embodiments the β-sheet peptide comprise an amino acid sequence having at least 90% or at least 95% identity with the sequence of QQKFQFQFEQQ (SEQ ID NO. 6); QQKFQFQFHQQ (SEQ ID NO. 7); FKFEFKFE (SEQ ID NO. 8); KFQFQFE (SEQ ID NO. 9); QQRFQFQFEQQ (SEQ ID NO. 10); QQRFQWQFEQQ (SEQ ID NO. 11); FEFEFKFKFEFEFKFK (SEQ ID NO. 12); QQRFEWEFEQQ (SEQ ID NO. 13); QQXFXWXFQQQ (Where X denotes ornithine) (SEQ ID NO. 14); FKFEFKFEFKFE (SEQ ID NO. 15); FKFQFKFQFKFQ (SEQ ID NO. 16); AEAKAEAKAEAKAEAK (SEQ ID NO. 17); AEAEAKAKAEAEAKAK (SEQ ID NO. 18); AEAEAEAEAKAKAKAK (SEQ ID NO. 19); RADARADARADARADA (SEQ ID NO. 20); RARADADARARADADA (SEQ ID NO. 21); SGRGYBLGGQGAGAAAAAGGAGQGGYGGLGSQG (SEQ ID NO. 22); EWEXEXEXEX (Where X=V, A, S, or P) (SEQ ID NO. 23); WKXKXKXKXK (Where X=V, A, S, or P) (SEQ ID NO. 24); KWKVKVKVKVKVKVK (Where X=V, A, S, or P) (SEQ ID NO. 25); LLLLKKKKKKKKLLLL (SEQ ID NO. 26); VKVKVKVKVDPPTKVKVKVKV (SEQ ID NO. 27); VKVKVKVKVDPPTKVKTKVKV (SEQ ID NO. 28); KVKVKVKVKDPPSVKVKVKVK (SEQ ID NO. 29); VKVKVKVKVDPPSKVKVKVKV (SEQ ID NO. 30); VKVKVKTKVDPPTKVKTKVKV (SEQ ID NO. 31); Fmoc-FF; Fmoc-GG; Fmoc-FG; KKSLSLSLSLSLSLKK (SEQ ID NO. 32); or YTIAALLSPY (SEQ ID NO. 33). In certain embodiments the β-sheet peptide comprise an amino acid sequence having at least 90, 91, 92, 93, 94, 95, 96, 97, 98, 99, or 100% with the sequence of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, or SEQ ID NO. 33.

In certain embodiments the β-sheet peptide comprises an amino acid sequence consisting essentially of, consisting or comprising the sequence any of the peptides of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, or SEQ ID NO. 33. In certain embodiments the β-sheet peptide is Q11—QQKFQFQFEQQ (SEQ ID NO. 6).

In certain aspects, the invention provides a method of preparing a nanofiber complex composition, comprising mixing the following:

a) a plurality of non-R-sheet peptide tags, wherein a non-R-sheet peptide tag is attached to a compound; and
b) a plurality of D-sheet peptides, under conditions that allow one or more of the non-β-sheet peptide tags to undergo transition from a non-R-sheet structure to a R-sheet structure, thereby preparing a nanofiber complex composition that forms a R-sheet structure comprising the transitioned non-R-sheet peptide tags and β-sheet peptides. Any one of the β-sheet peptides described in the invention could be used.

In certain aspects, the invention provides a method of inducing an immune response in a subject, comprising administering to the subject any one of the inventive compositions of the invention.

In certain embodiments, the method further comprises administering an adjuvant.

### BRIEF DESCRIPTION OF DRAWINGS

The patent or application file contains at least one drawing executed in color.

FIG. 1 shows overview of experiments described in Example 1.

FIG. 2 shows a short segment of a Q11 nanofiber (left) illustrating the fibrillized Q11 domain (blue), and appended epitopes projecting from the surface of the nanofiber (green and red). The full nanofiber is hundreds of nanometers long (Right, TEM of Q11 nanofibers bearing PADRE T-cell epitopes and a B-cell epitope from TNF (Example 1 reference 1).

FIG. 3 shows adjusting the amount of T-cell epitopes within nanofibers tunes the strength of antibody responses against B-cell epitopes. Mice were immunized with peptide formulations consisting of a fixed molar ratio of 1 mM TNFQ11 (B-cell epitope) and progressively increasing amounts of PADREQ11 T-cell epitope. Mean±SD is shown. *p<0.05, **p<0.001 compared to 0 mM T-cell epitope by ANOVA. See (Example 1 reference 1) for additional details.

FIG. 4 shows design of the CH505 TF ch.SOSIP. The portion of the CH505 TF gp120 that was N-terminal to the alpha-5 helix was transplanted into the BG505 SOSIP sequence. The stabilized CH505 TF ch.SOSIP formed trimeric proteins as shown by 2D class averages of negative stain electron microscopy images.

FIG. 5 shows B cell calcium flux induced by stabilized SOSIP trimer. Calcium flux was measured in B cells from C57BL/6 (left) or CH103 UCA light and heavy chain knock-in mice (right). Transitional and mature B cells are indicated with red and blue curves respectively. Mature B cells lacked a response because they are anergic. Arrows indicate the time of addition of anti-IgM (top) or stabilized CH505 TF ch.SOSIP (bottom).

FIGS. 6A-6C show creation of CH505 TF SOSIP ferritin nanoparticles by sortase-A conjugation. FIG. 6A. Diagram of CH505 TF SOSIP trimer showing the orientation of sortase A linkage to ferritin. The N-terminus of the conjugate is shown on the right. FIG. 6B. A model of a CH505 env SOSIP ferritin particle with 8 Env trimers displayed, based on ferritin and SOSIP trimer crystal structures. FIG. 6C. Negative-stained EMs of CH505 TF SOSIP ferritin nanoparticles created by sortase-A conjugation. The number of trimers per particle varies because of the variability of orientation of the particles on the EM grid.

FIG. 7 shows schematic of supramolecular SOSIP trimer synthesis. The peptides G15-Q11 and PADRE-Q11 are synthesized individually using solid-phase peptide synthesis. These are self-assembled into nanofibers in PBS. Subsequently, SOSIP trimers with LPETGG C-terminal tags will be attached to the nanofibers using sortase-A to produce the final supramolecular SOSIP trimer.

FIGS. 8A-8C show optimized synthesis of gp120-nanofiber conjugates. To preserve antigenicity and maximize mAb binding to gp120 (FIG. 8A) we tested >25 formulations and improved from undetectable nanofiber coupling and poor mAb binding (FIG. 8B) to 95% coupling efficiency with mAb binding comparable to unmodified gp120 (FIG. 8C).

FIG. 9 shows infant rabbits were vaccinated at 2 and 5 weeks of age with 15 g of gp120 combined with either 1) no adjuvant, 2) 2% squalene emulsion (SE), or 3) 800 ug Alum (n=5/group). Unvaccinated littermate(s) serve as negative controls (n=3). Serum gp120-IgG concentration was determined by ELISA at week 5 and 7. Two weeks after the second immunization (7 weeks of age), the magnitude of the vaccine-elicited antibodies was significantly higher in the gp120+SE group than in the unvaccinated and gp120 alone groups (p=0.02 and 0.04 respectively). Kruskal-Wallis with Dunn's multiple comparison, multiplicity adjusted P value reported.

FIGS. 10A-10E show enhancement of vaccine-elicited antibodies in mice immunized with a nanofiber-conjugated gp120 vaccine. (FIG. 10A) Higher magnitude of 1086.c gp120-specific antibodies in mice immunized with 50 g Q11 nanofiber-conjugated gp120 (gp120-Q11) than in mice immunized with gp120 alone. (FIG. 10B) After a single immunization, mice immunized with gp120-Q11 in the presence of STR8S-C adjuvant have higher magnitude antibody response than those immunized with gp120+STR8S-C. (FIG. 10C) The addition of the T cell epitope PADRE increases the immunogenicity of the gp120-Q11 vaccine. (FIG. 10E) Glycoprotein antigen conjugated to peptide nanofibers potentiates B cell responses. (FIG. 10D) Square symbol shows gp120 conjugated to Q11 nanofibers. Circle symbol shows gp120 envelope.

FIG. 11 shows rabbits immunized with stabilized CH505 transmitted/founder Env trimers, develop auto-logous neutralizing antibodies. Neutralizing antibody titer is shown as reciprocal dilution of serum that inhibits 50% of virus replication for rabbits immunized with unstabilized, open SOSIP Env trimers (n=4) or with stabilized closed SOSIP Env trimers (n=8). Horizontal bar represents the geometric mean of the group.

FIG. 12A shows neutralization of tier 2 viruses by 2 CH505 SOSIP vaccinated rabbits. FIG. 12B shows immunogenicity study in rabbits.

FIGS. 13A-13C show antibodies elicited by HIV Env vaccination in rabbits and rhesus monkeys can bind to human FcR and recruit FcR bearing effector cells. Magnitude of binding to human FcRs in plasma from rabbits and rhesus macaques immunized with 1086c. gp120 (FIG. 13A). The ratio of binding to FcR3a/FcR2a (FIG. 13B) and the ability to recruit human NK cells (determined by area scaling analysis in the ADCC-GTL assay) was higher in rabbits as compare to monkeys (FIG. 13C).

FIG. 14A shows pilot immunogenicity study in infant rhesus macaques. FIG. 14B shows titration of SHIV CH505 in infant rhesus macaques. Six infant macques were originally challenged by bottle feeding with a dose of 5.10×10⁵ TCID₅₀/day. Uninfected animals (n=5) were subsequently challenged orally under sedation with a weekly dose 6.8×10⁵ TCID₅₀. Finally, the remaining uninfected monkey was challenged with increased virus doses until infection.

FIG. 15 shows challenge study in infant rhesus macaques

FIG. 16 shows infant rhesus macaques born to HIV vaccinated dams or RSV vaccinated controls were orally challenged with SHIV 1167ipd34 at 6 weeks of age. While there was no association between passively acquired antibodies and risk of infection, a negative association was observed between % of activated CD4+ T cells and number of challenge required for infection.

FIG. 17 shows one non-limiting embodiment of conjugation of gp120 to Q11 self-assembled peptide nanofibers

FIGS. 18A and B show sequence of CH505TF.6R.SOSIP.664.v4.1_C_SORTAv3. In FIG. 18B, LPSTGG is one embodiment of sortase linker. In another embodiment, the linker is LPXTG₁₅. In another embodiment, the linker is LPXTG₁₅-beta tail. Underlined is the signal peptide.

FIGS. 19A and 19B show that Q11 nanofiber can provide higher degree of multivalency. FIG. 19A shows images of a fiber synthesized by the schematic shown in FIG. 19B.

FIGS. 20A-20C show the antigenicity of 1086c. gp120 is generally preserved following Q11 conjugation. The ability of a panel of HIV Envelope-specific monoclonal antibodies (mAb) to bind to the Q11-conjugated 1086C gp120 (FIG. 20B) and to the unconjugated 1086c gp120 (FIG. 20A) was evaluated by ELISA and Biolayer interferometry (BLI) (FIG. 20C). Equivalent binding was observed for CD4 binding site mAb VRC01 and for the V2-specific mAb CH58, whereas the CD4 binding site mAb B12 and the V3-specific mAb CH22 showed reduced but detectable binding to gp120-Q11.

FIGS. 21A and B show Gp120-Q11 induced higher magnitude antibody responses than the unconjugated gp120 vaccine. FIG. 21A shows C57BL/6 mice were immunized with gp120-Q11 (n=4) or the unconjugated gp120 (n=5) at a dose of 50 μg of gp120 at week 0, 2, 5 and 11. FIG. 21B shows antibody responses to 1086c gp120 were then measured by ELISA. After three vaccine doses, the magnitude of the binding antibody response was higher the gp120-Q11 than in the unconjugated gp120 groups. The response remains higher in the gp120-Q11 group after the fourth vaccine dose. Overall, there was a statistically significant difference in antibody levels between the two groups (p=0.047, two factors repeated measures ANOVA).

FIG. 22 shows Gp120-Q11 induced heterologous antibody binding responses earlier and of higher magnitude than the unconjugated vaccine. The breadth of the vaccine-elicited antibody response was measured after the third and the fourth immunization against a cross-clade panel of HIV envelope gp120 and gp140 using a binding antibody multiplex assay. The log percentile of the mean fluorescence intensity (MFI) area under the curve was used to construct a heat map and the AUCs were compared between the two groups of animals using a Mann Whitney U test. Immunization scheme is shown in FIG. 21A.

FIGS. 23A and 23B show gp120-Q11 adjuvanted with STR8S-C induced higher magnitude antibody responses than STR8S-C adjuvanted unconjugated gp120. FIG. 23A shows C57BL/6 mice were immunized with gp120-Q11 (n=5) or unconjugated gp120 adjuvanted (n=5) with the squalene based Toll like receptor 7/8 and 9 agonist STRES-C at a dose of 15 μg of gp120 at week 0, 10 and 26. FIG. 23B shows anti-1086c gp120 antibodies were measured by ELISA. Anti-gp120 antibodies were detected as early as 2-4 weeks after the first vaccine dose and were significant higher in the group immunized with the adjuvanted Q11 conjugated vaccine (p=0.017). STR8S-C is a vaccine adjuvant that stimulates TLR7/8 and TLR9.

FIG. 24 shows gp120-Q11 adjuvanted with STR8S-C induced higher magnitude heterologous antibody binding responses than STRE8 S-C adjuvanted unconjugated gp120. The breadth of the vaccine-elicited antibodies was measured after the primary and secondary immunizations and at the time of the third immunization, using a binding antibody multiplex assay. After the second immunization animals immunized with gp120-Q11/STR8S-C had higher binding to the clade B and AE envelope proteins than animals immunized with gp120/STR8S-C. Immunization scheme is shown in FIG. 23A.

FIGS. 25A-25C show that higher density of gp120 on Q11 fibers induced higher antibody response. FIG. 25A shows that to test the impact of antigen spacing on anti-gp120 immune responses, gp120-Q11 nanofibers with different densities of gp120 were formulated. FIG. 25B shows mice were immunized with equal doses of 15 microg of gp120 and a 100 microL injection volume. For “high loading” nanofibers, vaccines were formulated with nanofibers containing 7.5 microM gp120, which is equivalent to 1 antigen spaced between 250 β-sheet peptides. “Low loading” fibers were formulated with 1.6 microM gp120, which is equivalent to 1 antigen spaced between 1200 β-sheet peptides. Finally, unassembled antigens were tagged with a non-fibrillizing peptide (C-SGSG-QQKPQPQPEQQ). The total dose of the antigen was maintained constant between groups. Groups of 10 animals were immunized with the different vaccine constructs at a dose of 15 μg of gp120 at week 0 and week 4. FIG. 25C shows gp120-specific antibodies were measured by ELISA. P=0.007 *gp120^{high}-Q11 vs gp120^{low}-Q11; P=059 *gp120^{low}-Q11 vs gp120-CP3Q11. Two-factor repeat measure ANOVA, time*vaccine interaction term.

FIG. 26 shows that CD4 T cell epitopes can be easily added onto gp120-Q11 nanofibers. PADRE-Q11 is synthesized using solid phase peptide synthesis and then co-assembled with Cys-Q11 at predetermined ratios. Using this strategy, T cell epitopes can be added into the nanofiber formulations at precise ratios. PADRE is a nonnatural peptide epitope able to bind most of the human HLA-DR types and mouse I-A^{b} type.

FIG. 27 shows that Gp120-Q11 PADRE induced higher antibody titers after the first vaccine dose. Vaccine induced antibody responses were measured by ELISA. After the first vaccine dose the group immunized with gp120-Q11 PADRE had higher titers of anti-gp120 antibodies than animals immunized with gp120-Q11. However, this effect was no longer observed after the second immunization.

FIG. 28 shows sortase-mediated CH505 gp120-Q11 conjugation.

FIGS. 29A-29H show the antigenicity of the Q11-sortase conjugated CH505 gp120 evaluated using ELISA by comparing the binding of a panel of mAbs to the conjugated and unconjugated gp120. The binding of all the tested mAbs was comparable between the conjugated and unconjugated gp120 for all the HIV-specific mAbs including the CD4 binding site mAb Ch31 and CH235.12 (FIGS. 29A and 29B), the V2 glycan dependent mAbs PG9 and PG16 (FIGS. 29C and 29D), the V3 glycan dependent mAb PGT126, and the V3-specific mAb Ch22 (FIGS. 29E, 29F, 29G, and 29H).

FIG. 30 shows nanofiber morphology of CH505 gp120-Btail/Q11 assessed by TEM.

FIG. 31 shows strategy of β-tail mediated incorporation of SOSIP envelope.

FIGS. 32A-32B show data for conjugation per strategy described in FIG. 31.

### DETAILED DESCRIPTION

Some of the challenges of development of HIV vaccine include: extended diversity of the HIV-1 population; difficulty at inducing broadly neutralizing antibodies (bnAbs) through vaccination. BnAbs can neutralize most of the HIV strains; where passive immunization with bnAbs protects non-human primates from infection. (R. Shibata et al., 1999); BnAbs are only found in 10-50% HIV+ patients years after infection. (P. Hraber et al., 2014), currently no vaccine has been able to induce bnAbs in human or in animal models.

A pediatric vaccine against HIV would have a significant clinical impact, because more than 150,000 infants are infected with HIV every year globally, despite the availability of antiretroviral drugs to prevent mother-to-child transmission. Antiretroviral (ARV) interventions fall short via a number of mechanisms, including poor maternal adherence, fetal/infant toxicities, acute maternal infection during pregnancy and breastfeeding, transmission of drug-resistant strains of virus, and an inherent residual risk of transmission even in mothers on optimal ARV regimens. These limitations of ARV strategies have revealed that development of a pediatric vaccine against HIV will be required to eliminate mother-to-child transmission. Moreover, there is also a critical need for preventive measures to reduce adolescent HIV infections that occur following sexual debut. A pediatric HIV vaccine that offers protection in infancy and durable protective immunity through sexual debut could significantly reduce both infant and adolescent HIV infections.

The infant immune system poses challenges for vaccination but represents an excellent opportunity for developing molecularly engineered vaccines. In particular, the neonatal immune system is limited by a reduced ability to provide T-cell help, which results in poor somatic hypermutation of antibodies and inadequate antibody affinity. In addition, induction of long-term immunity following infant immunization usually requires several vaccine boosts. Surprisingly, recent studies have indicated that HIV gp120 vaccinated children develop higher magnitude and more durable antibody responses compared to the same vaccine regimen in adults. Moreover, HIV-infected children develop neutralization breadth earlier than adults, suggesting that it may be easier for a vaccine to elicit this type of response in children than in adults. Yet, there have been no HIV vaccine approaches specifically developed for the infant immune system. Immunization with native-like HIV envelope constructs constitutes a leading strategy for elicitation of neutralization breadth, but despite advances in stabilization and production of native-like HIV-1 trimers over the last decade, typical vaccination strategies with HIV-1 Envelope SOSIP trimer products have been disappointing in their ability to raise broad and potent virus-neutralizing activity. Novel vaccine approaches are therefore critically needed in order to achieve persistent, effective anti-HIV antibody responses and broad virus neutralization.

We have recently developed a supramolecular peptide nanofiber-vaccine platform (Q11) that can provide durable antibody responses with tunable titers. To address the limitations of the early life immune system and improve the immunogenicity of HIV envelope trimers, we propose to utilize this novel multivalent supramolecular nanofiber vaccine platform to design an engineered vaccine containing 1) optimized quantities of a synthetic T-helper epitope (PADRE) and 2) multimeric scaffolded SOSIP Env trimers of the HIV-1 transmitted/founder envelope CH505. In certain embodiments, the PADRE-nanofiber conjugated HIV-1 CH505 SOSIP trimer vaccine (P-Q11 CH505 trimer) will enhance the magnitude and potency of tier 2 virus neutralization responses in small animal and infant non-human primate (NHP) models, and will be protective against homologous SHIV challenge in an infant NHP challenge model.

In certain aspects, the invention provides methods to develop and assess the antigenicity a supramolecular nanofiber-based PADRE-scaffolded CH505 SOSIP HIV-1 Env trimer. In certain embodiments, the antigenicity of CH505 SOSIP HIV Env trimer is preserved in the nanofiber platform.

In certain aspects, the invention provides methods to define the immunogenicity of the P-Q11CH505 trimer vaccine in neonatal rabbits and infant rhesus macaques in comparison to that of CH505 SOSIP Env trimer alone. In certain embodiments, the P-Q11CH505 trimer vaccine will elicit a higher magnitude of antibodies than CH505 SOSIP Env trimer alone, including tier 2 virus neutralization and non-neutralizing effector antibody responses.

In certain aspects, the invention provides methods to determine the ability of the P-Q11CH505 trimer vaccine to protect against low dose oral SHIV challenge in an infant nonhuman primate model of late postnatal transmission via breastfeeding. In certain aspects, the invention provides that infant rhesus monkeys immunized with the P-Q11CH505 trimer vaccine will be protected against infection following low dose SHIV oral challenge as compared to unvaccinated animals.

This novel pediatric HIV vaccine strategy could overcome the challenges of infant vaccination, while taking advantage of the immunologic and practical benefits of early life immunization. Notably, the addition of T-cell epitopes will stimulate neonatal T-cell responses to provide the T cell help require to drive affinity maturation and tier 2 neutralizing antibody development; while the nanofiber platform will yield durable B-cell responses. Importantly, because this vaccine system is fully synthetic and modular, it has manufacturability advantages over other vaccine platforms and it can be systemically optimized for diverse immunization settings.

Multivalency is critical in activating B cells—because of BCR cross-linking.

In certain embodiments, multivalent antigen presentation on ferritin-based HIV vaccines increases the neutralization against heterologous strains. (K. Sliepen et al., “Presenting native-like HIV-1 envelope trimers on ferritin nanoparticles improves their immunogenicity” Retrovirology volume 12, Article number: 82 (2015)). In some embodiments, in the ferritin system the number of antigen presented is limited, and it could be difficult to control the stoichiometry of antigens or epitopes. In certain aspects, the invention provides engineered vaccine which overcome the major challenges of HIV vaccine development. In certain aspects, the invention provides nanofiber compositions comprising HIV-1 envelopes.

In certain aspects the invention provides Q11 based nanofiber compositions comprising HIV-1 envelopes. In certain aspects the invention provides a Q11 nanofiber a vaccine platform that induces more potent humoral responses than unconjugated gp120. In certain aspects the invention provides that a gp120-Q11 immunogen leads to increase anti-gp120 antibody magnitude and breadth of responses. In certain aspects, the invention provides that Q11-conjugated gp120 induces higher antibody magnitude than vaccine with gp120 alone. In certain aspects the invention provides that Q11-conjugated gp120 induces can increase the antibody response in the presence of adjuvant. In certain aspects the invention provides that gp120-Q11 induced even higher antibody magnitude in the presence of STR8S-C adjuvant. In certain aspects, the invention provides that the gp120 density on Q11 affect the antibody response. In certain aspects, the invention provides that higher density of gp120 on Q11 fibers induced higher antibody response. In certain embodiments, the invention provides that CD4 T cell epitopes can be easily added onto gp120-Q11 nanofibers. In certain aspects, the invention provide that additional CD4+ T cell epitopes on gp120-Q11 can increase the magnitude and avidity of anti-gp120 antibodies.

In certain aspects the invention provides that Q11-conjugated gp120 increases the antibody magnitude and binding breadth; that innate immunity activated by TLR7/8 and TLR9 agonist augments the effect of Q11 nanofibers; recruitment of T cell help by PADRE-Q11 induces rapid humoral response at early stage.

In certain aspects, the invention provides that multivalency is important for the elevated antibody response induced by gp120-Q11---gp120-Q11 with different gp120 density.

Nanofiber complex technology is disclosed in U.S. Pat. No. 9,200,082, and references #23 and #24 in Example 1, which contents are herein incorporated by reference in their entirety. Non-limiting embodiments of optimized chemistry and linkers used in the conjugation of the gp120 envelopes are disclosed in Example 1 and 1A, Example 2 and Example 3.

Self assemblies which can be modular, self-adjuvanating, and/or define are described in the art. These include but are not limited to: beta-sheet nanofibers; peptide polymer gels, helical fibrillar assemblies, assembling proteins, peptide amphiphiles, etc. See e.g. Hudalla et al., Nature Materials 13: 829-36 (2014), Rudra et al., PNAS, 107:622-7 (2010), Wen et al., ACS Nano, in press (2017), Rudra et al., Biomaterials 31:8475 (2010), Chen et al., Biomaterials 34:8776 (2013), Pompano et al., Adv Healthc Mater 3:1898 (2014), Wen et al., Curr Opin Immunol. 35:73 (2015), Trent A et al., AAPS J, 17:380 (2015), Black M et al., Adv Mater 24:3845 (2012), U.S. Pat. No. 9,200,082. Fibrilixing peptides are also known: peptide epitope-QQKFQFQFEQQ. These can form chemically defined nanofibers. Coil29 system is an example of a alpha-helical nanofibers. See e.g. E. H. Egelman et. al, Structure 2015, 23, 280, Y. Wu et al., ACS Biomater Sci&Eng 2017, 3, 3128, which contents are herein incorporated by reference in their entirety

Supramolecular assemblies are self-adjuvanting. See e.g. Rudra J S et al., PNAS, 107:622-7 (2010), Rudra et al., ACS Nano 6(2) 1557 (2012); Wen et al., ACS Nano, 10(10) 9274-9286 (2017); Rudra et al., Biomaterials, 33(27), 6476 (2012); Chen et al., Biomaterials, 34(34), 8776 (2013); Pompano et al, Adv Healthc Mater 3(11), 1898 (2014); Vigneswaran et al., JBMR A 104, 1853 (2016); U.S. Pat. No. 9,200,082, which contents are herein incorporated by reference in their entirety.

Peptide nanofibers raise durable antibody responses. See e.g. Y. Wu et al., ACS Biomater Sci & Eng 2017, 3, 3128. Peptide assemblies are non-inflammatory. See e.g. J. Chen, R. Pompano et al. Biomaterials, 2013 34, 8776; Pompano et al, Adv Healthc Mater 2014 3(11), 1898. An example is the use in Anti-TNF immunotherapy. See e.g. Mora Solano et al., Biomaterials 2017 149, 1-11. Adjustable titers could be based on nanofiber content. Mora Solano et al., Biomaterials 2017 149 1-11, which contents are herein incorporated by reference in their entirety.

The invention is directed to compositions comprising a supramolecular vaccine for HIV, its use and methods of making such.

Sequences/Clones

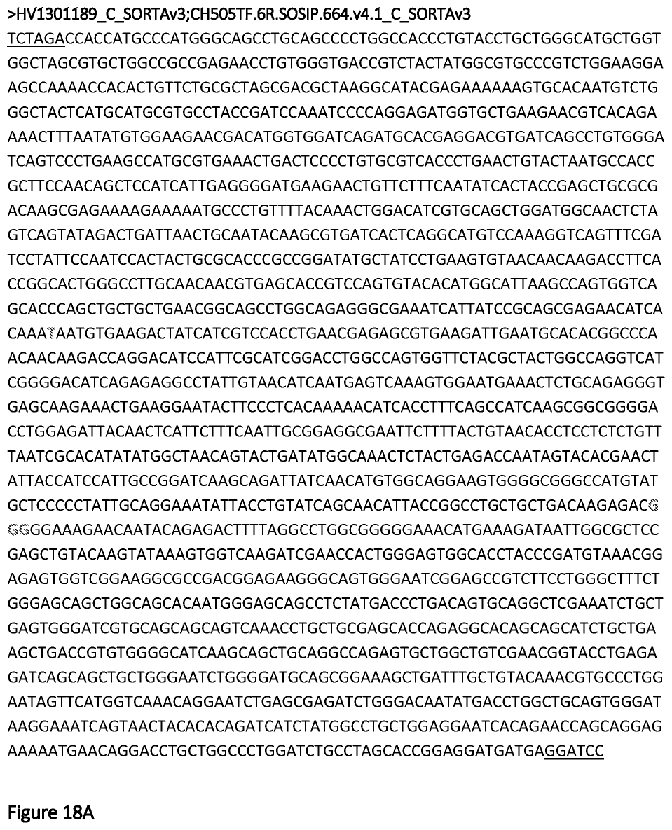

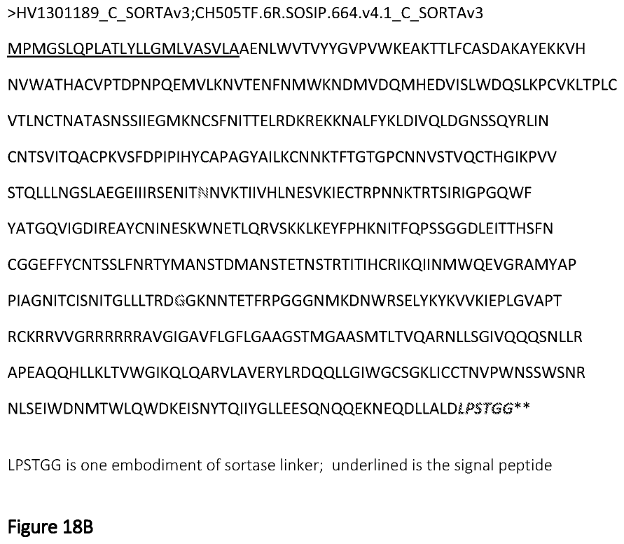

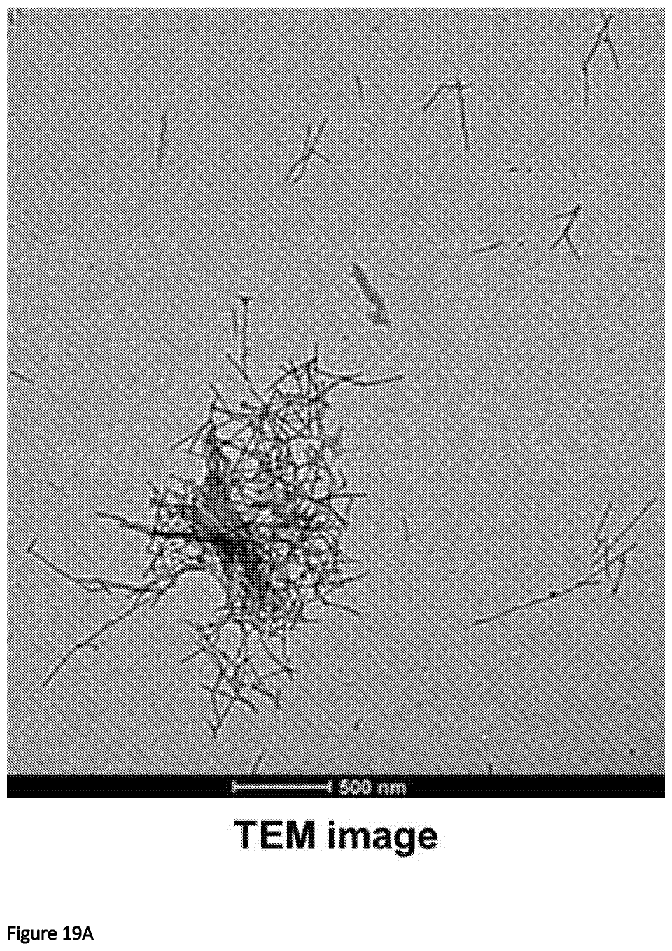

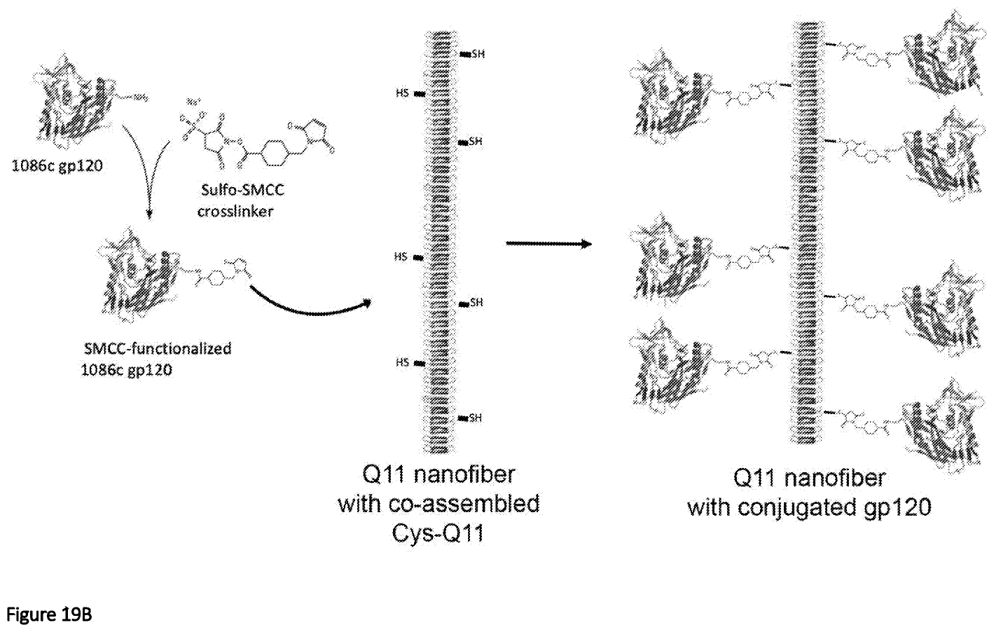

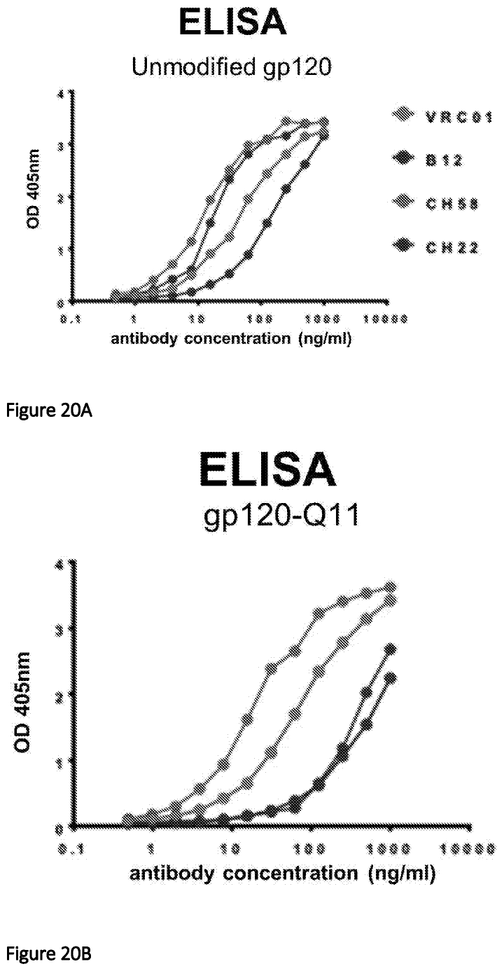

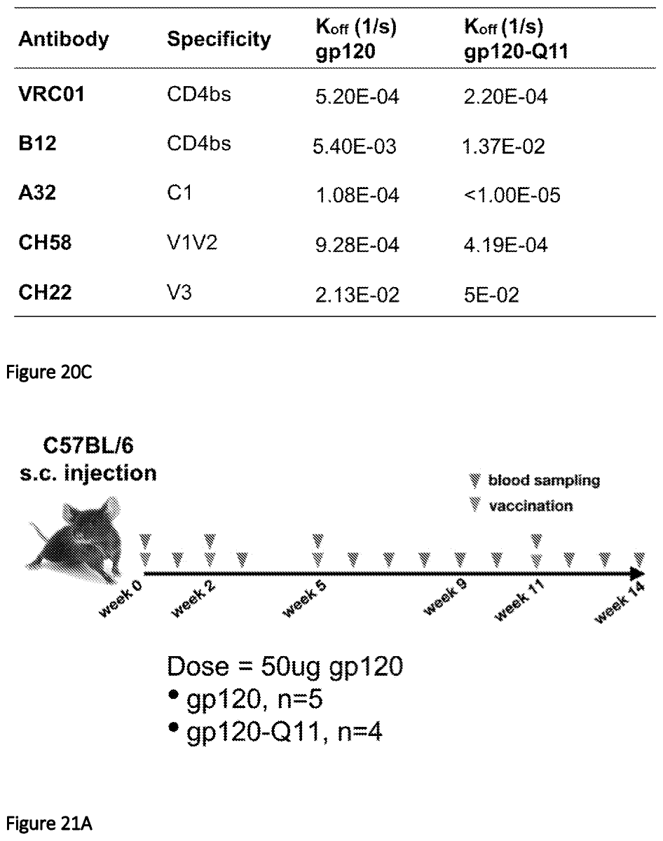

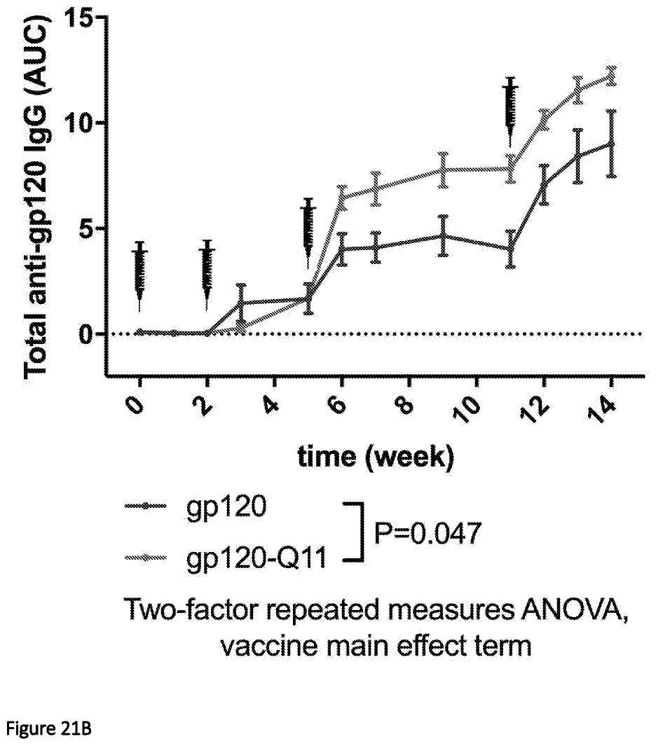

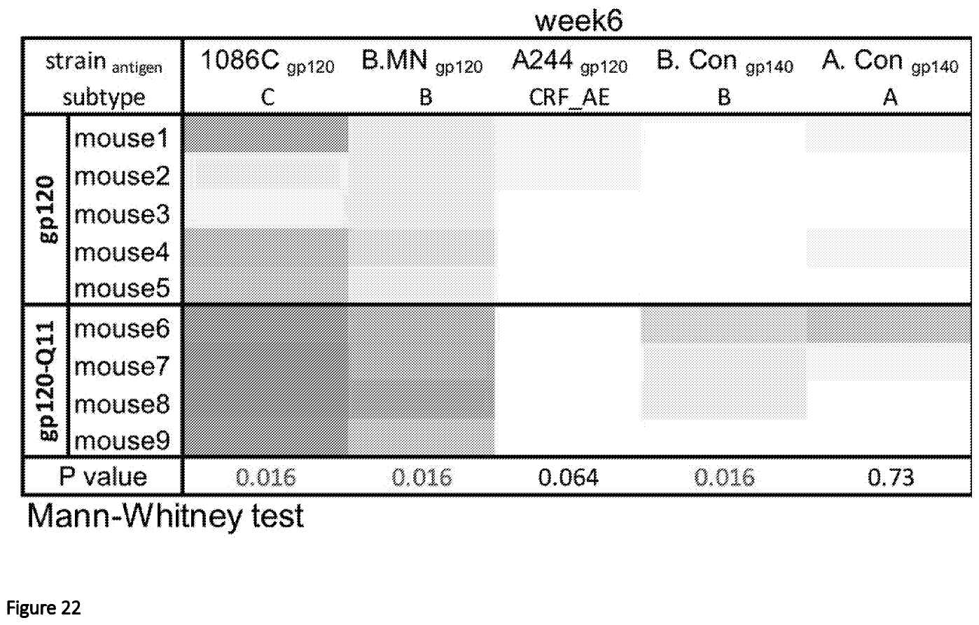

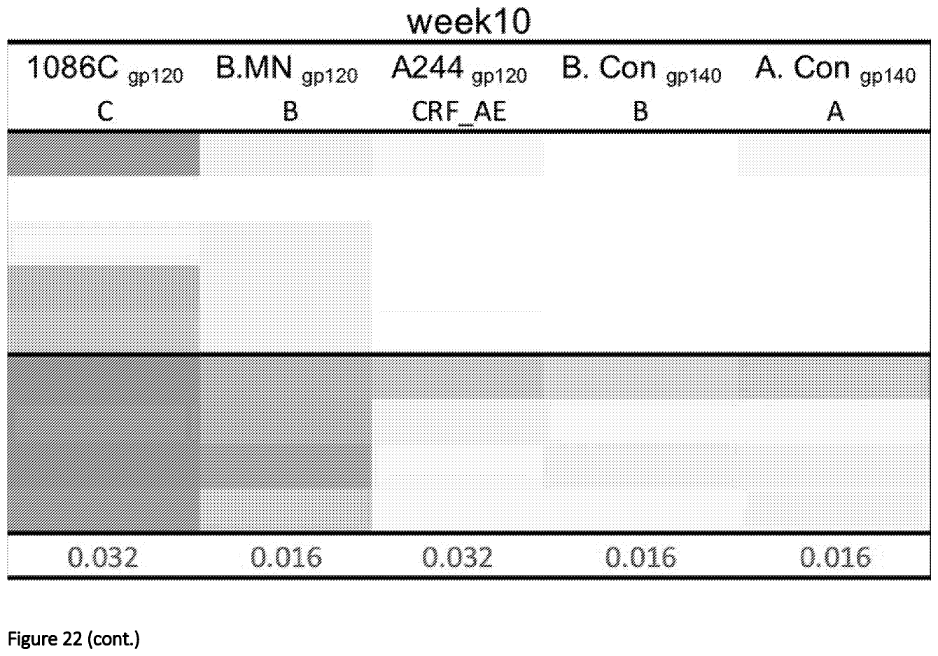

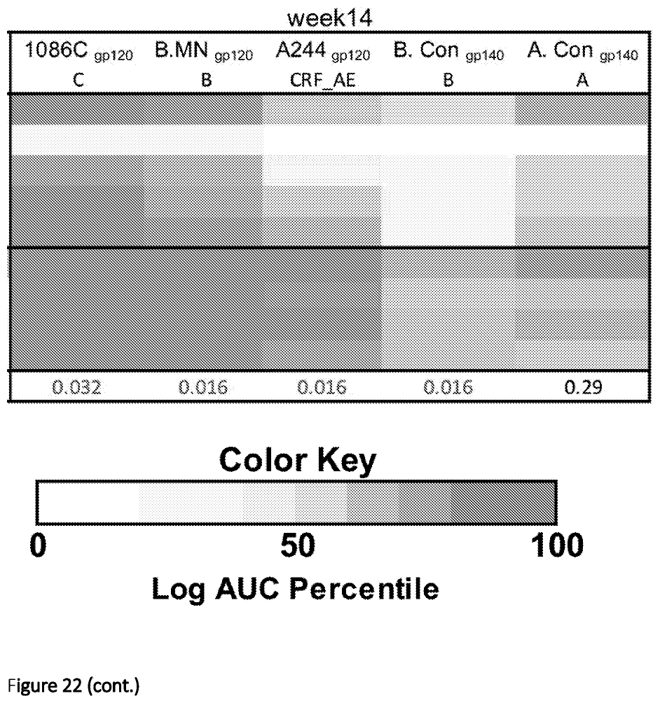

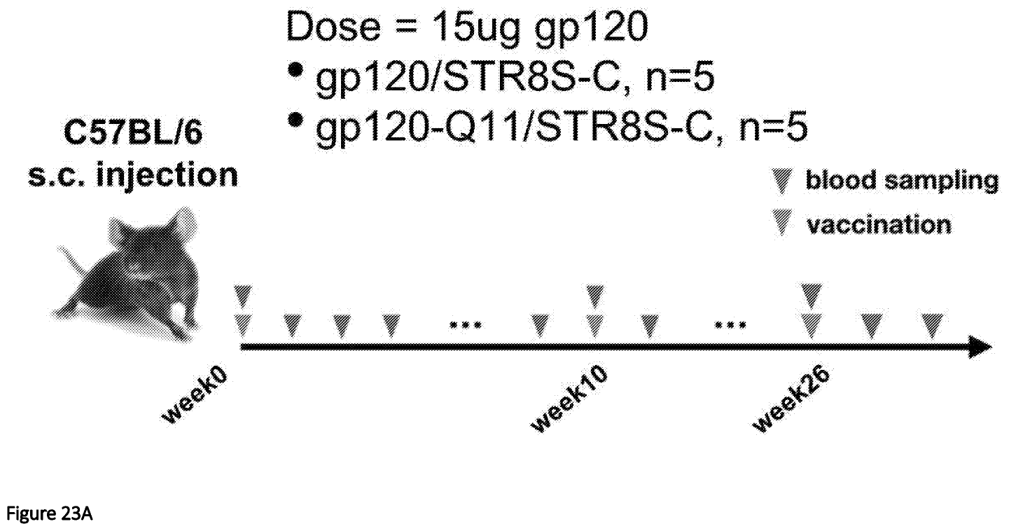

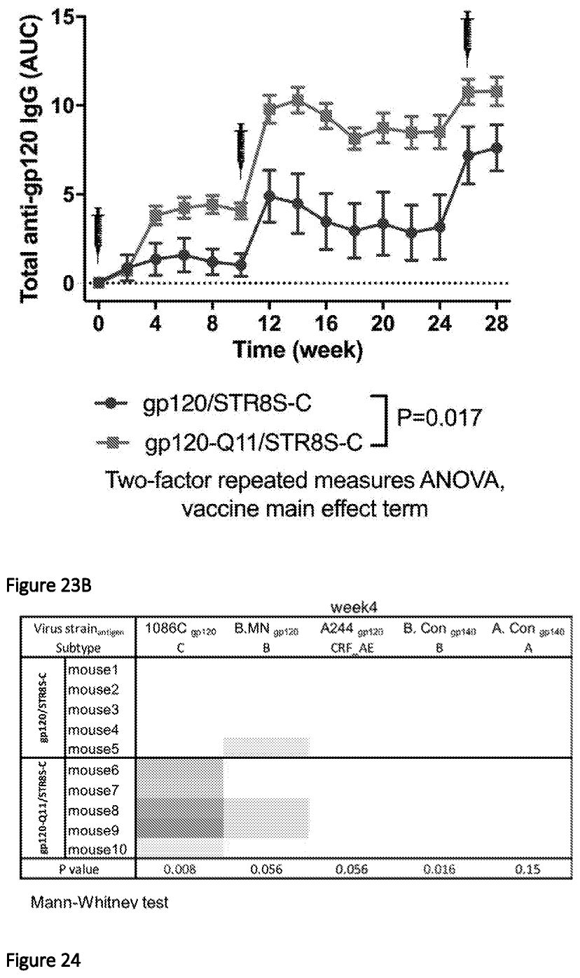

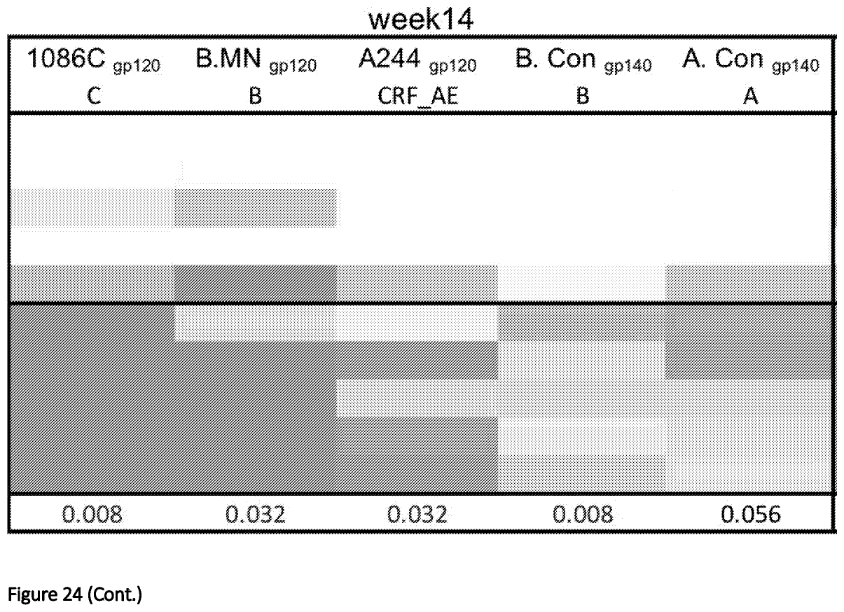

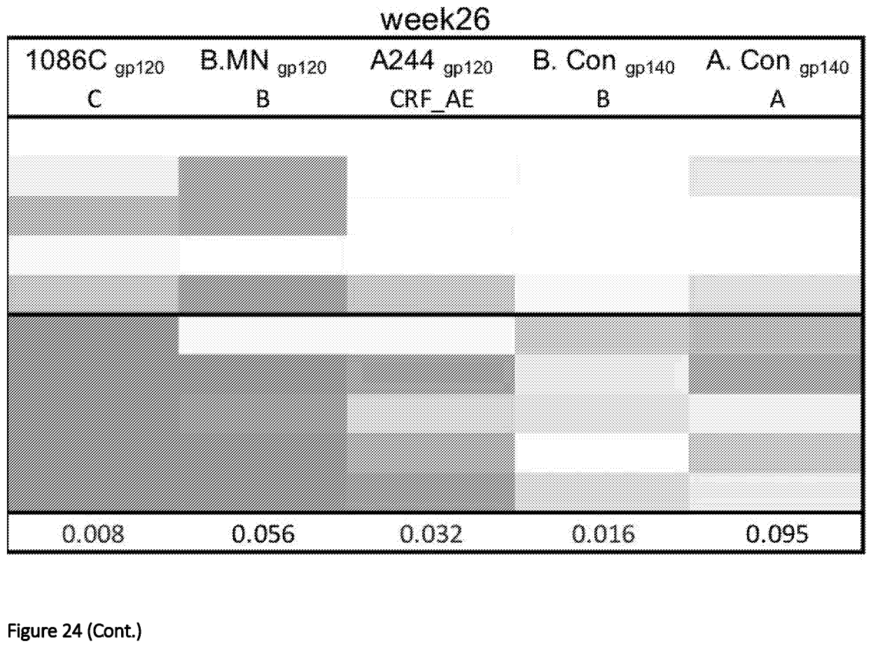

In certain embodiments, the envelope is gp120 envelope 1086.C. See e.g. US Publication 20140248301 which discloses a gp140 design of this sequence. The gp120 design of the envelope 1086.C. In certain embodiments, the envelope is trimer based on CH505 T/F see instant Example 1 and FIGS. 18A-B, and WO/2017/15180, supra. Additional sortase A linkers are also contemplated.

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41—named as gp140ΔCFI (gp140CFI), gp140 Envs with the deletion of only the cleavage (C) site and fusion (F) domain—named as gp140ΔCF (gp140CF), gp140 Envs with the deletion of only the cleavage (C)—named gp140ΔC (gp140C) (See e.g. Liao et al. Virology 2006, 353, 268-282), gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

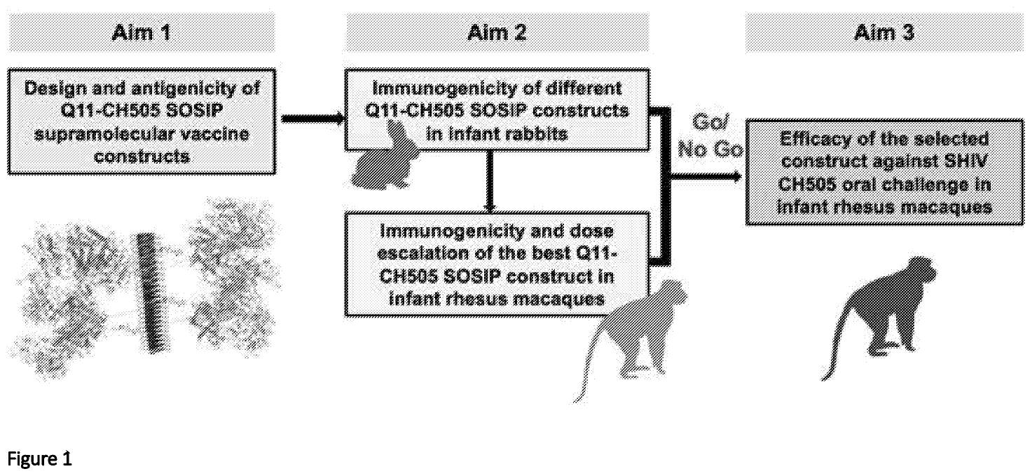

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR.” See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleave efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. April; 87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR is changed to ERVVEREKE, and is one example of an uncleaved gp140 form. Another example is the gp140C form which has the REKR site changed to SEKS. See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVHNVWATHACVPTDPNPQE . . . (rest of envelope sequence is indicated as “ . . . ”). In other embodiments, the delta N-design described for CH505 T/F envelope can be used to make delta N-designs of other CH505 envelopes. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence could be used. In some embodiments the leader sequence is the endogenous leader sequence. Most of the gp120 and gp160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA). Most of the chimeric designs include CD5 leader sequence. A skilled artisan appreciates that when used as immunogens, and for example when recombinantly produced, the amino acid sequences of these proteins do not comprise the leader peptide sequences.

Any suitable HIV-1 envelope, in any envelope design, could be conjugated to Q11 fibers. In certain embodiments, the envelope is conjugated using sortase A reaction.

The immunogenic compositions can be administered in any suitable regiment for prime and boost. Such regimens could comprise administering of any other suitable HIV-1 immunogen. In certain embodiments, the immunogenic compositions are administered to infants or young adults.

Dosing of proteins and nucleic acids can be readily determined by a skilled artisan. A single dose of nucleic acid can range from a few nanograms (ng) to a few micrograms (g) or milligram of a single immunogenic nucleic acid. Recombinant protein dose can range from a few g micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramuscular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to, alum, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen. In certain embodiments, TLR agonists are used as adjuvants. In other embodiment, adjuvants which break immune tolerance are included in the immunogenic compositions.

### Example 1

A vaccine is critically needed to eliminate pediatric HIV and generate protective HIV immunity prior to sexual debut. Pediatric HIV continues to be a public health concern in middle and low income countries, and despite the availability of antiretroviral drugs (ARV), more than 150,000 infants become infected through mother-to-child transmission every year (1). ARV interventions fall short via a number of mechanisms, including poor maternal adherence, acute maternal infection during pregnancy and breastfeeding, transmission of drug-resistant strains of virus, and an inherent residual risk of transmission even in mothers who receive ARV (2-5). In addition, the number of new HIV infections among adolescent girls and young women in sub-Saharan Africa remains exceptionally high: in 2015, 450,000 young women (aged 15-24 years) became infected with HIV (1, 6), and young women have three times more infections than their male counterparts. A pediatric HIV vaccine that offers protection in infancy and durable protective immunity prior to sexual debut could significantly reduce infant, adolescent, and life-long HIV infections.

Immunization in early life has to overcome limitations of the infant immune system including: 1) a reduced ability to provide T-cell help, which results in poor somatic hypermutation (SHM) of antibodies and inadequate antibody affinity, and 2) the need for several vaccine boosts to achieve durable immunity (reviewed in (7)). Because of these known limitations, it is generally thought that infants are not a suitable target population for HIV vaccine development. Nevertheless, in several settings, infants have been reported to develop comparable or higher immune responses than adults following vaccination. Notably, we have recently reported that infants immunized with a MF59 adjuvanted HIV gp120 vaccine developed higher magnitude antibody responses than adults immunized with the same vaccine, whereas comparable responses were observed between adults and infants immunized with an Alum adjuvanted HIV vaccine (8). Thus, our results suggest that adjuvants differently modulate immune responses in adults and infants. Similarly, recent studies have demonstrated that the response of newborn immune cells to most candidate adjuvants is functionally distinct from that of adults (reviewed in (9)). As vaccine strategies will benefit from being tailored to the early-life immune system, young children represent an important population for the development of molecularly engineered HIV vaccine approaches.

A major goal for HIV immunization is the induction of antibodies capable of neutralizing the majority of circulating viruses. These broadly neutralizing antibodies (bnAbs) emerge in approximately 20 to 30% of HIV-1 infected adults several years after infection (10-12), but have proven difficult to elicit by vaccination. This may be partially due to the fact that antibodies elicited by Env protein vaccination tend to bind poorly to the Env trimer at epitopes associated with bnAbs. It is therefore believed that immunization with native like Env trimers may be required for bnAb induction. Studies investigating the ability of Env trimers to induce neutralizing antibody responses have found that some constructs are able to induce tier 1 and autologous tier 2 neutralization, but infrequent heterologous tier 2 neutralization in rabbits and guinea pigs and to a lesser extend in rhesus macaques (13). Interestingly, our team recently observed infrequent, low to moderate levels of tier 2 neutralization in rabbits immunized with a stabilized CH505 transmitted/founder SOSIP trimer (14). Thus, improvements on the current Env trimer-based immunization strategies are likely to be required in order to achieve neutralization breadth.

While current knowledge on neutralization breadth development in the setting of natural infection is mostly based on adult studies, recent studies have indicated that HIV-infected children may develop neutralization breadth earlier than adults. Milligan, Overbaugh et al., studied the development of cross-clade neutralization in 28 HIV infected children. They found that 20/28 children developed cross-clade neutralization, some as early as one year post infection (15). Similarly, Muenchhoff et al. reported that the frequency of broad neutralizers is higher among chronically HIV-infected children than in chronically infected adults (16). Interestingly, neutralization breadth in HIV-infected children is mediated by polyclonal antibodies (17), in contrast to adults in whom plasma neutralization is usually predominantly mediated by antibodies of one or two specificities (18). Moreover, pediatric bnAbs may have lower levels of somatic hypermutation than adult bnAbs from the same specificity with comparable breadth (19). Overall these data suggest that it could be easier to elicit neutralization breadth by vaccination in children than in adults and highlight the need to test these vaccine strategies in pediatric populations.

In certain aspects, the invention provides a multivalent supramolecular nanofiber vaccine platform to enhance vaccine responses. A supramolecular peptide nanofiber vaccine platform, Q11, that can provide durable antibody responses with tunable titers has been developed. We have previously shown that this system can elicit remarkably durable antibody responses of up to one year following a simple prime-boost regimen (20, 21), owing to the material's delayed degradation and extreme multivalency. We have also published strategies for installing and tuning the T-helper epitope content using the modular self-assembly afforded by the platform (22, 23), and have shown that the platform can function in the absence of additional adjuvants (21, 24). Because components of the vaccine spontaneously assemble into higher-order architectures in aqueous solutions, formulations can be readily modified without having to repeat chemical synthesis as is necessary for covalently constructed biomaterials (25). While supramolecular materials are receiving increasing interest for the design of vaccines and other active immunotherapies against infectious diseases such as influenza (24), MRSA (22), and malaria (20), they had not yet been used in the context of HIV vaccination. In preliminary experiments, we assessed the immunogenicity of a nanofiber-conjugated gp120 subunit vaccine in adult C57BL6 mice. The nanofiber subunit vaccine induced higher magnitude Env-specific antibodies than gp120 alone. Moreover, co-expression of gp120 with a synthetic T-Cell epitope (PADRE) on the nanofiber-led to a faster and more robustly binding antibody response. Thus, Q11 constitutes a versatile, potent, and engineerable vaccine platform that can be systematically adapted for specific immunologic settings.

Therefore, there is a critical need (1) for a pediatric HIV vaccine to protect against both breast milk transmission and adolescent sexual transmission; and (2) HIV Env trimer immunization platforms to achieve broad neutralization. Because the early-life immune system may be more amenable to the development of neutralization breadth than the adult immune system, such strategy should ideally be tested in the setting of early-life immunity. Without being bound by theory, the supramolecular nanofiber vaccine platform presents several advantages including 1) induction of long-lived immune responses; 2) antigenic persistence which could drive the maturation of the immune response through SHM; and 3) incorporation of a dominant T cell epitope that could help overcome the described poor T cell help in infants and contribute to the development of bnAb responses. We therefore propose to design a supramolecular nanofiber conjugated HIV SOSIP trimer vaccine (P-Q11 CH505 trimer) and then test its immunogenicity/efficacy in infant animal models (FIG. 1).

In certain aspects, invention provides a supramolecular nanofiber Q11 vaccine platform for use with HIV immunogens. The vaccine platform itself has recently been developed (22, 24). The Q11 self-assembling peptide system has been explored as the basis for vaccines and immunotherapies in mouse models for a variety of diseases and conditions, including malaria (20), influenza (24, 26), bacterial infections (22), and chronic inflammation (23), and was shown to be capable of raising durable, high-titer antibody responses and T-cell responses against a variety of antigens without requiring additional adjuvant. However, the Q11 platform has not yet been explored for the design of a HIV vaccine.

In certain aspects, the invention provides the first primate immunogenicity assessment of this vaccine platform. It will be the first time that this supramolecular peptide vaccine will be tested in primates. The proposed work thus represents a critical test of the concept that supramolecular peptide vaccines can be immunogenic in higher order species.

In certain aspects, the invention provides s scaffolded, multimeric, and self-adjuvanted native-like trimer design to build on the moderate success of native trimer immunogens. A stabilized CH505 TF ch.SOSIP trimer antigen has been developed. In this work will develop bioconjugation techniques to attach this antigen to supramolecular peptide nanofibers, and in some embodiments to deliver it in the context of a global CD4+ T cell epitope (PADRE).

In certain aspects, the invention provides a modular and tunable nature of the Q11 vaccine platform. Because each of the supramolecular construct's components can be individually synthesized and combined in precise and tunable stoichiometries, and because the supramolecular assemblies are nanofibers hundreds of nanometers long, we will be able to optimize the antigen loading and T-helper epitope content across a wide range, in addition to optimizing the dose, boosting regimen, and adjuvant. Such tunability is not common among vaccine platforms, making the Q11 platform and our approach for optimizing it inventive.

In certain aspects, the invention provides, design and preclinical development of an HIV vaccine for the early-life immune system. Despite known differences in the adult and the infant immune system, HIV vaccine candidates have been routinely tested in adult preclinical studies and only a handful are eventually tested in pediatric settings. Our approach of designing a vaccine construct to specifically overcome known limitations of the early-life immune system and to first testing this construct preclinically in infants.

In certain aspects, the invention provides designs of a molecularly engineered pediatric HIV vaccine based on CH505 SOSIP HIV-1 Env trimer.

In certain aspects, the invention provides methods to develop and assess the antigenicity a supramolecular nanofiber-based PADRE-scaffolded CH505 SOSIP HIV-1 Env trimer.

In certain embodiments, the invention incorporates the Q11 system of fibrillizing peptides and the optimized envelope designs, including but not limited to CH505 SOSIP HIV-1 Env trimers. Without being bound by theory, the hypothesis is that combining these components will preserve the antigenicity of the CH505 SOSIP trimer and provide a vaccine capable of raising durable antibody responses in infant macaques. Our proposed work will represent the first trial of the Q11 vaccine system in primates, providing not only a critical proof-of-concept that a supramolecular form of the SOSIP trimer can improve antibody titers and durability, but also a demonstration that the Q11 platform is immunogenic in primates, which could be applied to vaccines for other diseases in follow-on work.

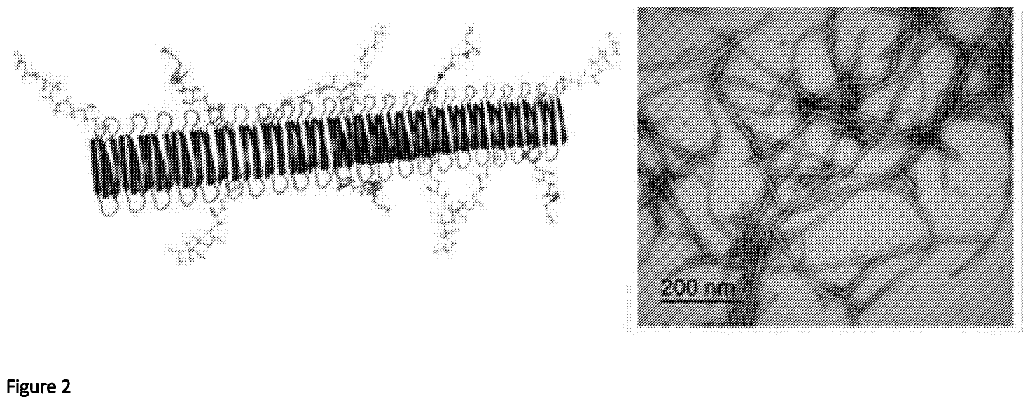

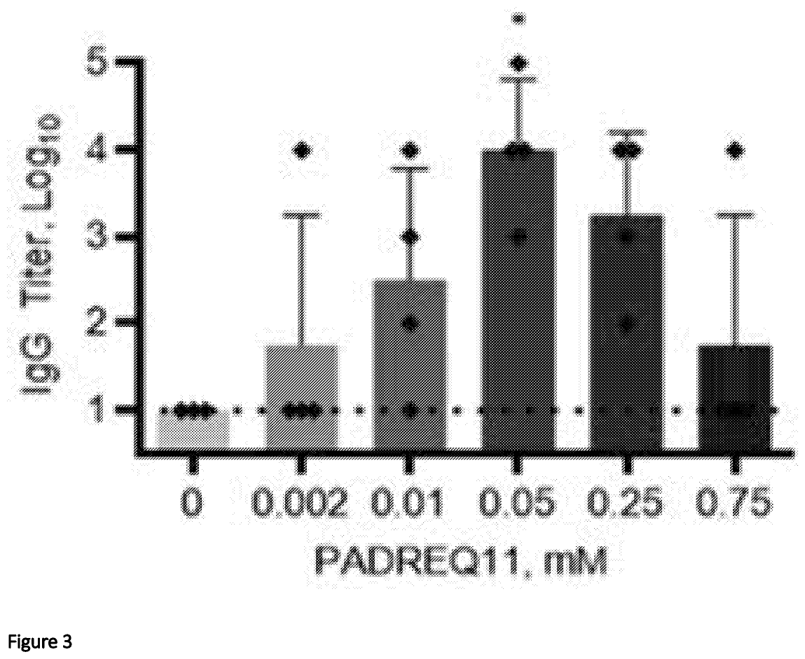

The Q11 system is composed of a short synthetic peptide, one embodiment is QQKFQFQFEQQ. When dissolved in water and subsequently added to buffered saline or fluids such as cell culture media or interstitial fluid, the peptide assembles into nanofibers containing hundreds to thousands of individual peptides (FIG. 2, showing Q11 nanofibers bearing PADRE T-cell epitopes and a B-cell epitope from TNF (23) (21). We have found that the assembly of the Q11 sequence is remarkably tolerant to the attachment of a variety of cargoes, including cell-binding ligands (27, 28), short peptide epitopes (22, 23, 26), or protein antigens (29, 30). We also discovered that peptide self-assemblies are self-adjuvanting; that is, capable of raising strong epitope-specific B-cell and T-cell responses without supplemental adjuvants (21). Multiple different Q11-appended peptides or proteins can be synthesized individually, combined in solution, and subsequently induced to assemble in a process that provides nanofibers with precise stoichiometric control over the constituents. Using this modularity, we have found that the ratio of PADRE T-cell epitopes to B-cell epitopes dramatically influences the titer and quality of the B-cell and T-cell responses induced (FIG. 3) (22, 31). Here, we will apply this system to produce highly multivalent nanofibers containing SOSIP trimers and PADRE T-cell epitopes.

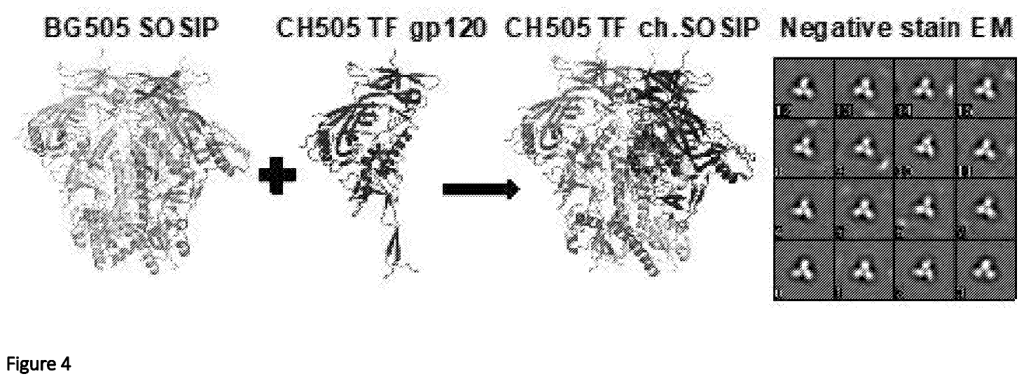

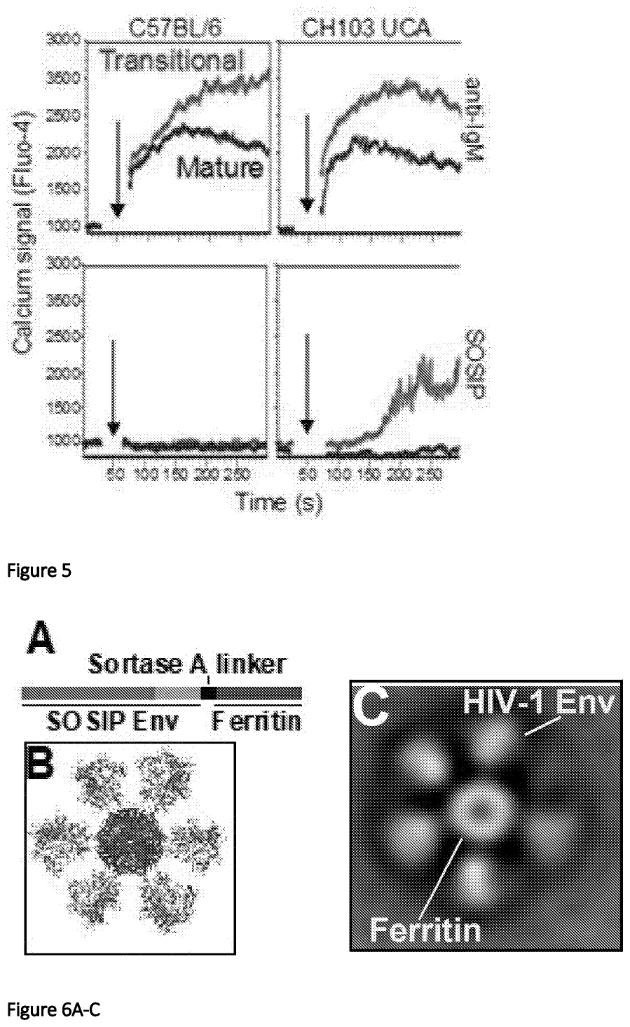

We have recently designed a stabilized CH505 TF ch.SOSIP trimer targeting the germline of CH103, a CD4 binding site bnAb identified in an HIV-infected patient that developed broadly-neutralizing activity (32). Initial attempts to express a CH505 TF SOSIP found it to be unstable resulting in very little trimeric envelope protein (33). To improve trimer formation, a chimeric SOSIP (ch.SOSIP) was designed, in which the CH505 TF gp120 was used to replace the gp120 sequence of BG505 SOSIP (FIG. 4 and (14)). To stabilize the CH505 TF ch.SOSIP we introduced E64K and A316W mutations (34). The purified stabilized CH505 TF ch.SOSIP formed trimers as shown by negative stain electron microscopy (FIG. 4); was antigenic for broadly neutralizing antibodies directed against V2-glycan, V3-glycan, CD4 binding site, and gp120-gp41 interface; and lacked binding to non-neutralizing antibodies against the C1, V2, coreceptor binding site, and V3 regions. The addition of the E64K and A316W mutations into the CH505 TF ch.SOSIP eliminated antibody recognition of the V3 region and coreceptor binding site indicating the stabilized ch.SOSIP was not in the CD4-induced conformation (14). The SOSIP protein bound to the unmutated common ancestor (UCA) of the CH103 B cell lineage with 171 nM affinity, and the stabilized CH505 TF ch.SOSIP also engaged the CH103 UCA B cell receptor expressed on the surface of mouse B cells with sufficient affinity to induce calcium flux (FIG. 5).

Previous strategies for multimerizing SOSIP Env trimers have been successful but limited in their multivalency. The B cell receptor recognizes and internalizes low-affinity antigens at a greater magnitude when the low-affinity antigen is presented as a multimeric particle as opposed to monomeric protein in solution (35). In vivo, the multimerization of HIV-1 Env has improved neutralizing antibody titers in rabbits (36) and monkeys (37). We have developed methods for expressing and purifying the CH505 Env trimers as multimers using ferritin nanoparticles. Purification of SOSIP gp140-ferritin fusion proteins can be complicated by the presence of well-folded and poorly-folded trimeric Env on the same nanoparticle, so we developed a two-step ferritin assembly process where we first purified well-folded SOSIP gp140 trimers and separately purified ferritin nanoparticles. We then covalently linked the SOSIP to ferritin via short sortase-A linker peptides (FIG. 6). The presence of HIV-1 Env trimers on conjugated ferritin particles was confirmed with negative-stain electron microscopy. In sum, the prior development of the Q11 platform, the stabilized CH505 TF ch.SOSIP trimer, and the sortase-A bioconjugation strategy for attaching the trimer to multimeric carriers now enable us to design an even more highly multivalent supramolecular vaccine.

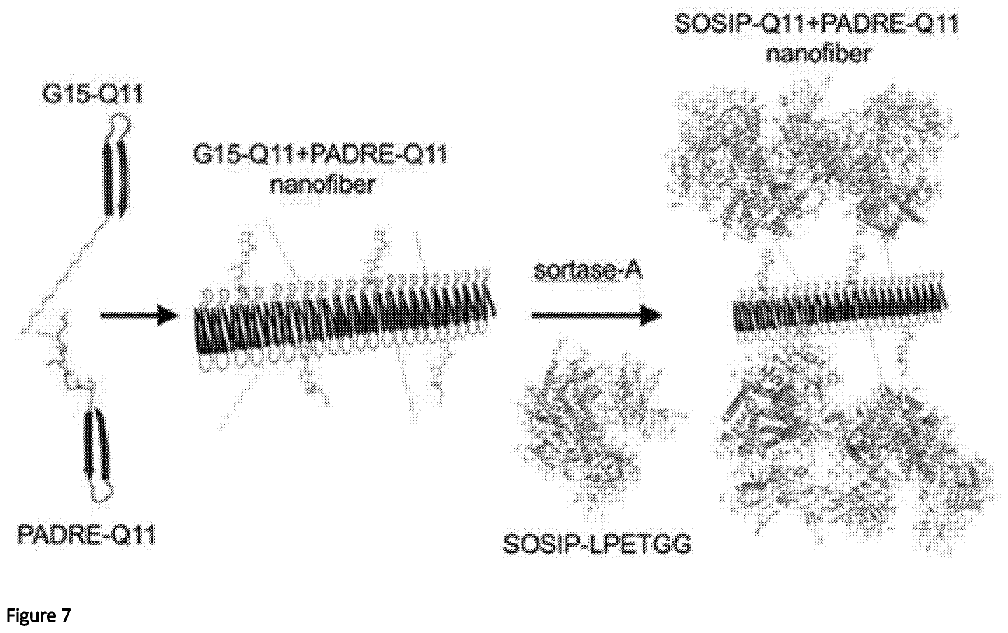

In certain aspects, the invention provides method for designing and producing a supramolecular SOSIP trimer vaccine. We will develop a supramolecular nanofiber vaccine by conjugating the previously optimized stabilized SOSIP trimer to self-assembled Q11 peptide nanofibers using sortase-A conjugation (synthesis scheme in FIG. 7). In short, peptides containing the Q11 self-assembly domain at the C-terminus and the sortase-A linker (Gly)₁₅ (G15) at the N-terminus will be will be synthesized as previously described using Fmoc-based solid phase peptide synthesis chemistry (38). Peptides will be purified to >95% purity using reverse-phase HPLC and stored as lyophilized powders. G15-Q11 will be assembled into nanofibers by adding aqueous solutions of 4 mM peptide to phosphate buffered saline (38), and nanofiber formation will be assessed using transmission electron microscopy (TEM, FIG. 2). We expect to observe that the sortase-A peptide will not impact self-assembly into fibers, as dozens of similar-sized peptides have been studied in the Q11 system before with uniform self-assembly behavior. In certain embodiments, we will also produce PADRE-Q11 (aKXVAAWTLKAAa-SGSG-QQKFQFQFEQQ, where “a” is D-alanine and “X” is cyclohexylalanine), containing the pan-DR T-cell epitope at its N-terminus. This peptide will be ultimately co-assembled within the nanofibers. As prior work has indicated that PADRE concentrations of 50-100 microM provided optimal T cell help, this will be the range of our initial concentrations studied. The stabilized CH505 SOSIP Env trimers containing C-terminal sortase tag LPSTGG will be expressed as has been achieved previously (FIG. 6). Trimers will be expressed in Freestyle293 cells and purified by affinity chromatography with trimer-specific bnAb PGT145. Trimeric gp140 will be isolated by size exclusion chromatography using a Superose 6 16_60 column. To produce the final vaccine, the PADRE-Q11, G15-Q11, and unmodified Q11 will be fibrillized together, and the SOSIP trimer will be conjugated by incubating with sortase-A overnight at room temperature. Reaction optimization may be necessary, but previous success conjugating the same trimer to ferritin nanoparticles bearing the same (GGG)₅ linker indicated that 75 μM trimer, 120 μM G15-linked nanofibers, and 100 μM sortase-A is an appropriate initial mixture. Fiber conjugation efficiency will be measured by centrifuging nanofibers and measuring residual protein in the supernatant. Antigenicity testing can be done when 1) The full fiber-linked SOSIP trimer forms nanofibers by TEM, 2) conjugation efficiencies of 80% or greater are achieved. In preliminary studies with monomeric gp120, such loading efficiencies were regularly achieved using heterobifunctional crosslinkers.

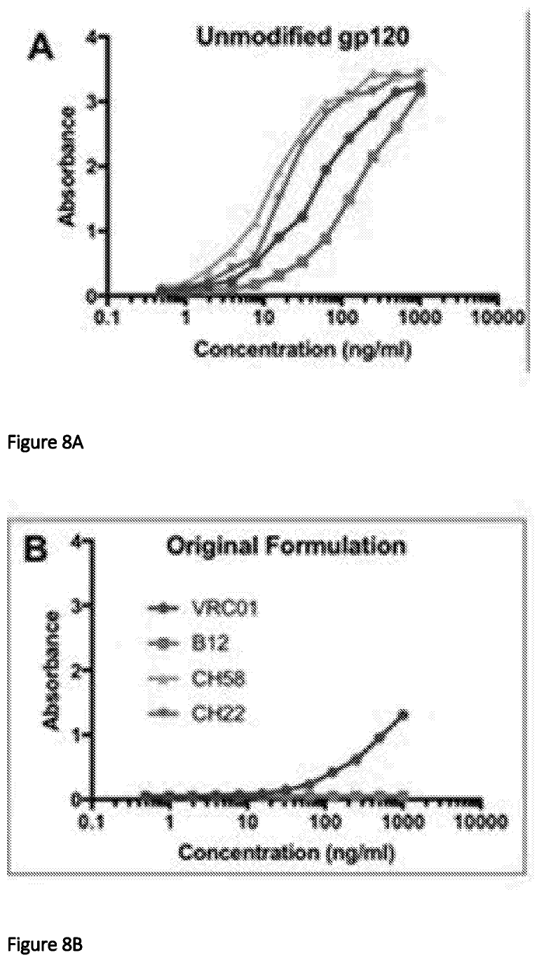

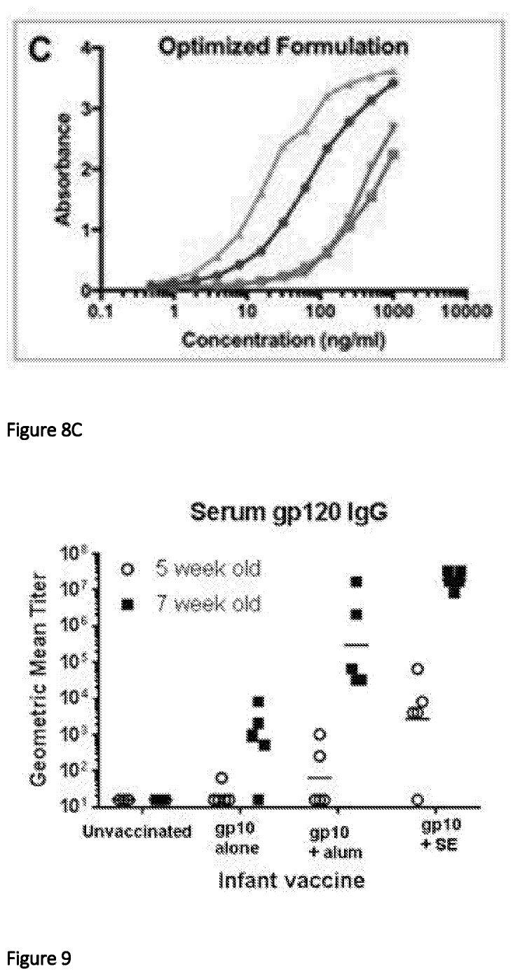

In certain aspects, the invention provides methods for testing the antigenicity of supramolecular Env vaccines. We have synthesized a simplified version of our proposed multimeric vaccine, tested its antigenicity, and studied its immunogenicity in mice. Briefly, we synthesized Q11, Q11 terminated with a single cysteine residue, and PADRE-Q11; co-assembled them into nanofibers; and conjugated the cysteine thiol side chain to amines in gp120 using SMCC, an amine-reactive and thiol-reactive heterobifunctional crosslinker. We optimized various linker lengths, linker chemistries, and reaction stoichiometries to achieve efficient nanofiber conjugation of the gp120 while preserving its antigenicity. Using the monoclonal antibodies VRC01, B12, CH58, and CH22 we assessed the antigenicity of key sites of the protein by ELISA. We progressively improved protein antigenicity such that the optimized formulation (FIG. 8C) showed highly similar antibody binding compared to the unmodified gp120 (FIG. 8A). This pilot-study material was subsequently used to study immunogenicity in mice, described in Aim 2. In the proposed work, we will ensure that the trimers are in a closed conformation by assessing V2, V3, and C1 exposure as we have done in our previous publication (15). Moreover, we will measure the binding of a panel of monoclonal antibodies—known to bind CH505 SOSIP—to the P-Q11 CH505 trimer constructs by capture ELISA and Bilayer interferometry. In addition, the ability of the P-Q11 CH505 trimers to engage the B cell receptor of CH103 UCA knock-in will be measure using a calcium mobilization assay (FIG. 5).

Sortase-A bioconjugation was used to link the SOSIP Env-1 trimer to other nanoparticles such as ferritin. Without being bound by theory, there is a possibility that this process will be hindered by the presence of the peptide nanofiber. If poor conjugation is observed, we have a number of options for recourse. First, given that the Q11 peptide is fully synthetic, we could produce new peptides with longer or more hydrophilic linker sequences between the assembly domain and the sortase A linker peptide. Second, we have already shown that heterobifunctional crosslinkers can be used to attach monomeric gp120 to Q11 nanofibers, and that this strategy retains good immunogenicity of the antigen (FIG. 8). We could adapt this process for conjugating the full SOSIP trimer.

Provided are studies to define the immunogenicity of the P-Q11CH505 trimer vaccine in neonatal rabbits and infant rhesus macaques in comparison to that of CH505 SOSIP Env trimer alone

The nanofiber vaccine platform that we propose to use in this study may help overcome some of the limitations of the early-life immune system yet capitalize on some of its advantages towards developing bnAbs. While this platform has been used for different applications in the mouse model, it has not yet been tested for immunogenicity in primates. We therefore propose to first test the modular Q11 vaccine constructs in rabbits and then confirm immunogenicity in infant rhesus macaques. We selected rabbits as the small animal model to down-select vaccine constructs because: 1) Adequate volume of blood can be collected for diverse immune measurement; 2) Rabbits are a well described model in HIV vaccine development, 3) Immunization with native-like Env constructs have been demonstrated to induce autologous tier 2 neutralization in rabbits, but not in mice; 4) our group has previous experience working with infant rabbits (see FIG. 9) and 5) although they are more expensive than mice, rabbits are still relatively affordable. We hypothesize that the P-Q11 CH505 trimer vaccine will elicit a higher magnitude of antibodies than CH505 SOSIP Env trimer alone, including tier 2 virus neutralization and non-neutralizing effector antibody responses.

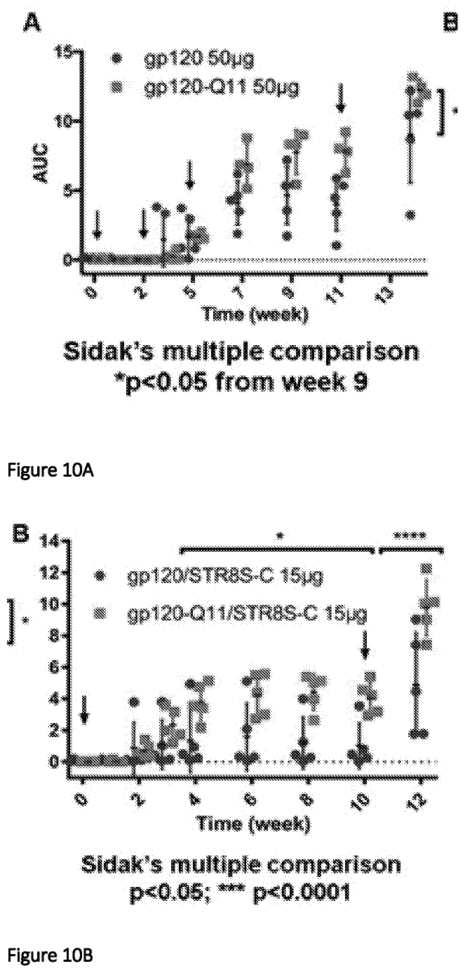

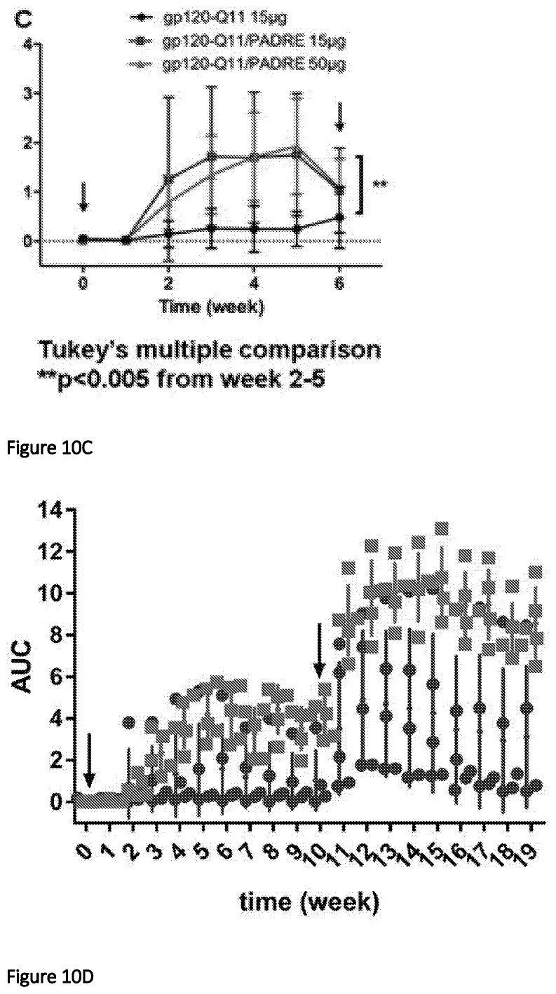

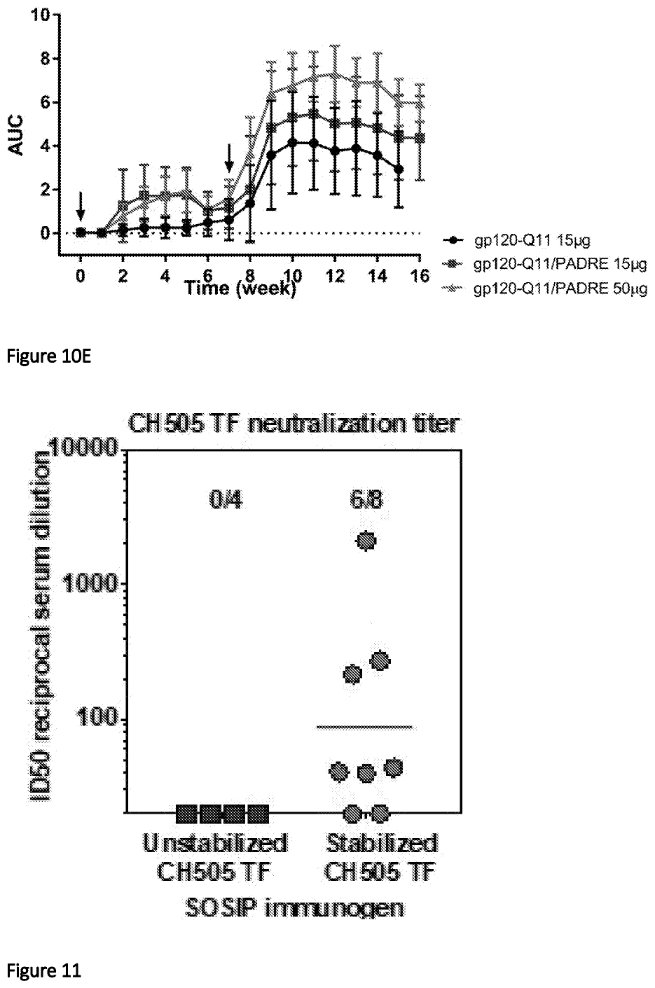

In certain aspects, the invention provides that immunization of adult mice with a gp120 nanofiber conjugated vaccine enhances antibody responses. We immunized C57BL6 adult mice (n=5) subcutaneously at week 0, 2, 5 and 11 with 50 μg of 1086.c gp120 conjugated to Q11 nanofiber (gp120-Q11) or with 1086.c gp120 alone. After the first 3 vaccines doses, gp120-Q11 induced higher antibody responses than gp120 alone (FIG. 10A). Further, mice that received gp120 alone had large individual-to-individual variability compared to those immunized with the gp120-Q11 formulation. We then assessed the immunogenicity of gp120-Q11 in the presence of an adjuvant. C57BL6 mice (n=5) were immunized with 15 g of gp120-Q11 or gp120 alone, along with STR8S-C, a TLR7/8 and TLR9 agonist adjuvant (FIG. 10B). With Q11 conjugation, a single dose of gp120 induced a higher antibody response than gp120/STR8S-C that lasted over 6 weeks. These results suggest that the presence of an external adjuvant does not nullify the adjuvancy of Q11 and highlights the possibility that adjuvants can function synergistically with Q11 nanofibers. As we had demonstrated previously that the co-assembling of a universal CD4+ T cell peptide epitope (PADRE) into the antigen-Q11 nanofibers can elicit higher titers possibly through the recruitment of T cell help, we also tested the effect of PADRE on the elicitation of anti-gp120 antibodies. To this end, we produced PADRE gp120-Q11 nanofibers containing 10 microg gp120, 100 microM PADRE, and 2 mM total peptide in the 100 microL formulation. Mice were immunized (n=10) with 15 g gp120 conjugated to Q11 or with either 15 g or 50 g gp120 conjugated to Q11 nanofibers containing PADRE. Vaccines containing PADRE induced a strong antibody response that rapidly reached a plateau 2 weeks post-vaccination and was sustained for at least 4 weeks (FIG. 10C).

This experiment exemplifies the modularity of the Q11 self-assembling peptide nanofiber as a vaccine platform for HIV. As the critical type(s) of immune response required for protection against HIV infection remain elusive, this modular feature renders Q11-nanofibers amenable for shaping the immune response against HIV antigens to target immune correlates as they are identified.

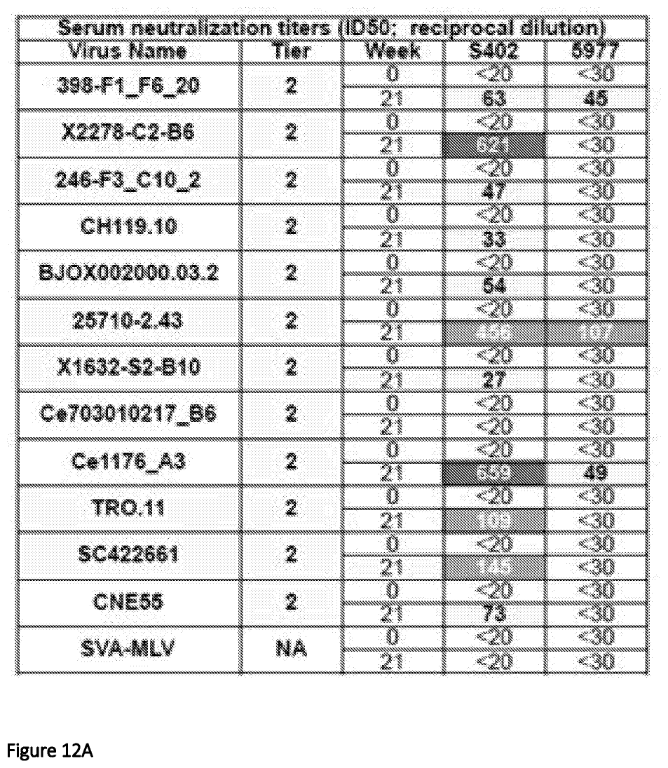

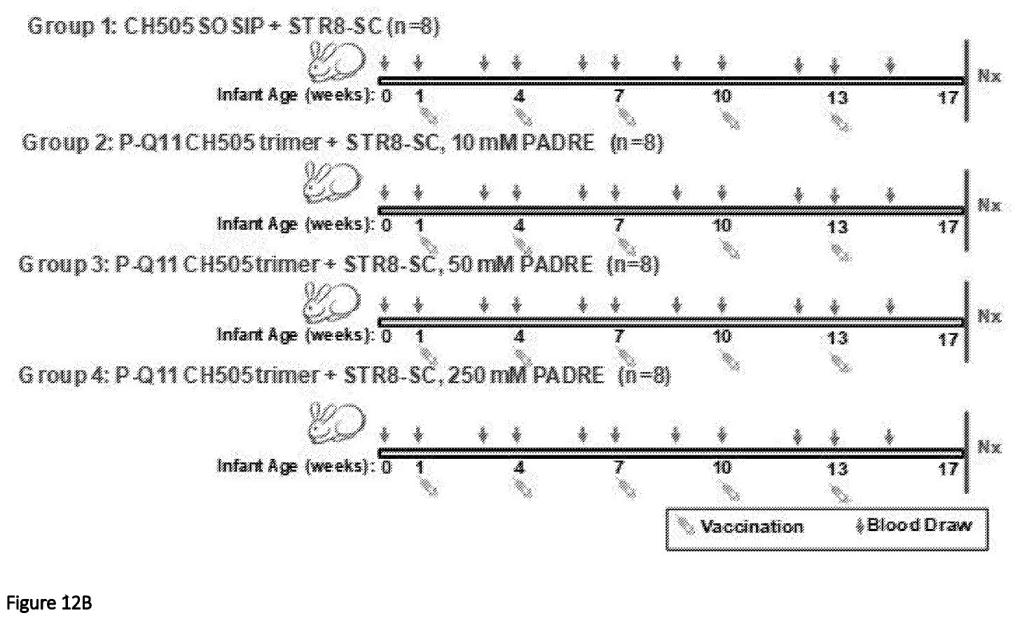

Elicitation of autologous and tier 2 neutralizing antibodies by CH505 SOSIP immunization in rabbits was reported previously (14). A stabilized CH505 TF SOSIP forms trimers and is antigenic for the UCA antibody of the CD4 binding site (CD4bs) CH103 bnAb lineage. The immunogenicity of the CH505 TF SOSIP trimer was tested in adult rabbits (14). Briefly, rabbits were immunized with either the unstabilized (E64/A316) or the stabilized (E64K/A316W) CH505 TF ch.SOSIP 4 weeks apart for a total of 6 vaccine doses. None of the rabbits immunized with the unstabilized CH505 TF ch.SOSIP developed autologous tier 2 neutralizing antibodies whereas 6 of 8 (75%) rabbits immunized with the stabilized SOSIP neutralized the autologous tier 2 CH505 TF virus (FIG. 11). The autologous tier 2 neutralizing antibodies were detectable in all animals after the fourth immunization but arose in two animals after only two immunizations. The autologous neutralizing antibodies were sensitive to mutation of the CD4 binding site and to the N160 glycosylation site. Sera from the rabbits were then tested against a panel of 12 heterologous tier 2 viruses. Two out of 8 rabbits immunized with the stabilized SOSIP generated heterologous tier 2 neutralizing antibodies (FIG. 12A). The sera from one of these rabbits (S402) was capable of neutralizing 11 of 12 tested isolates and the sera from the second animal (5977) neutralized 3 of 12 isolates. The specificities of the broad and potent serum neutralizing antibodies in rabbit S402 were mapped to the CD4bs and V3-glycan site. Overall these results indicate that the stabilized CH505 TF ch.SOSIP can elicit autologous and occasional heterologous tier 2 neutralizing antibodies targeting the CD4bs in rabbits. This study will test if conjugation of the stabilized CH505 trimer to the Q11 nanofiber would improve its immunogenicity and ability to induce tier 2 neutralizing antibody responses.

Contemplated are various immunogenicity studies in any suitable animal model. In non-limiting embodiments, the animals are rabbits.

Rabbit immunization schedule: Three groups of 8 neonatal rabbits will be immunized at 1, 4, 7, 10 and 13 weeks (FIG. 12B) afterbirth with either CH505 SOSIP Trimer with the STR8-SC adjuvant (group 1), or with P-Q11-CH505 trimer containing PADRE at a concentration of 10 microM (group 2), 50 microM (group 3) or 250 microM (group 4). PADRE concentrations were selected based on previous data generated in mice (see FIG. 3). Vaccine formulations will be administered into the subcutaneous space in keeping with established techniques for nanofiber vaccines (22, 23, 25). Sera will be collected before immunization and every 2 weeks after immunization for antibody measurement. The animals will be sacrificed 4 weeks after the last immunization.

Adjuvant and protein dose selection. STR8-SC is a squalene-based adjuvant containing oligoCpG as well as the TLR 7/8 agonist R848 (39). This adjuvant was selected because 1) previous work has demonstrated that TLR 7/8 and to a lesser extend other TLR agonists enhance immune responses in human and non-human primate neonates (9, 40); and 2) Previous data has indicated that while rabbit poorly respond to TLR7/8 agonists (41), they respond well to other TLR agonists; 3) the squalene-based MF59 adjuvant induced stronger Env-specific antibody responses than Alum following gp120 infant immunization (42) and 4) comparison of TLR agonist adjuvants in rhesus monkeys showed that STR8-SC induced the highest titers of binding and functional antibodies following HIV Env immunization (39). Animals will be immunized with a dose of 15 μg of protein. This dose was selected because 1) this dose was found to be optimal to induce Env-specific antibody responses in infants immunized with a MF59-adjuvanted HIV vaccine (42) and immunization of infant rabbits (FIG. 9) and infant rhesus macaques (43) with this dose induced robust antibody responses. In addition, a dose of 15 g of protein is cost-sparing, which would be beneficial for the manufacturing and implementation of an effective HIV vaccine.

Binding antibody responses to CH505 SOSIP trimer immunization in rabbits. The magnitude of the binding antibody response will be measured in the vaccine groups by capture ELISA as previously described (44). A polyclonal HIV-specific rabbit IgG reagent purified from a pool of plasma from HIV vaccinated rabbits (BIVIG) will be used as positive control. The avidity of the Env-specific antibodies will be measured as a surrogate for affinity maturation two weeks after each immunization. A single dilution of plasma selected based on the results from the titration experiment will be tested for binding to CH505 Env in the presence or in the absence of urea. The avidity index will then be calculated using the equation

$$\frac{OD ( urea ) X 100}{O D ( no urea )} .$$

Finally, we will use a binding antibody multiplex assay (BAMA) to define the breadth and epitope specificity of the vaccine elicited antibodies. The breadth will be assessed using a panel of cross-clade HIV Env glycoproteins whereas the epitope specificity will be assessed using peptide and Env constructs. In addition, we will measure the ability of the rabbit sera to inhibit the binding of bnAbs of known specificity (including bnAbs against the CD4 binding site, and against V1V2/V3 glycan-dependent epitopes) in a blocking ELISA.

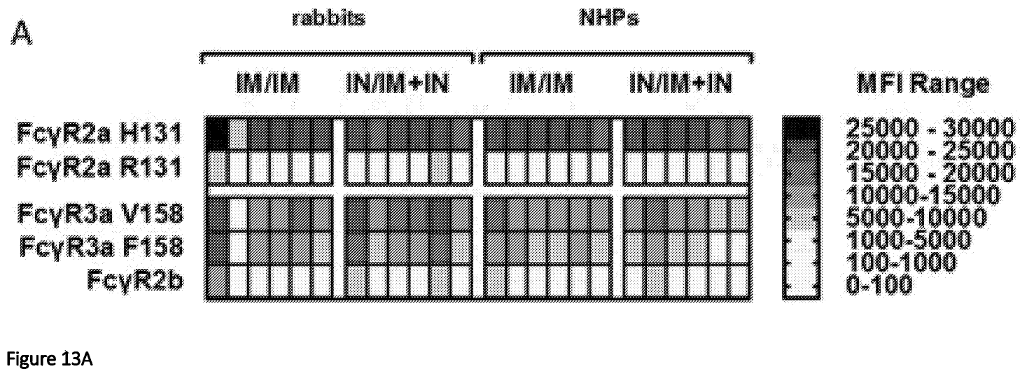

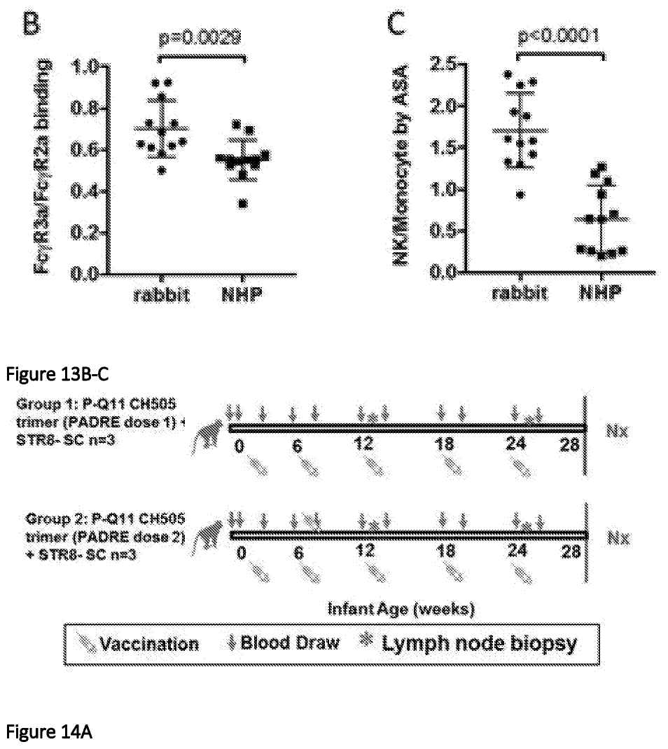

Measurement of functional antibody responses. One goal with this immunization strategy is to induce tier 2 neutralizing antibodies. We will therefore assess neutralizing antibodies using the TZM-bl assay against a panel of tier 2 viruses including the autologous HIV CH505 T/F virus and 4 heterologous tier 2 viruses. Neutralization of a control virus (SVA. MLV) will also be assessed to define non-specific neutralization activity. The primary time point for assessment of neutralizing antibody responses will be at necropsy because 1) the full immunization regimen may be required to induce the neutralizing antibodies and 2) we will have sufficient volume of blood to test activity against multiple virus strains. If neutralization is detected at the necropsy time point, we will assess previous timepoints to define when these neutralizing antibodies first developed. The epitope specificity of the autologous and heterologous neutralizing antibodies will be mapped using virus with mutations that abrogate the activity of known bnAbs using methods and assays known in the art. In addition to neutralization, we will also measure non-neutralizing antibodies because these responses have been linked to protection in the RV144 vaccine trial (45, 46) and NHP vaccine studies (47-49). Moreover, although sequential immunization with a DNA prime/CH505 gp120 boost vaccine did not induce broad neutralizing antibody responses in rhesus monkeys, this regimen was able to protect 67% of adult monkeys SHIV mucosal challenge, suggesting that protection was mediated by non-neutralizing antibodies. We will measure the ability of the vaccine-elicited antibodies to bind to HIV infected cells and mediate antibody dependent cell-mediated cytotoxicity (ADCC). In addition, we will assess the binding of vaccine-elicited antibodies to FcRs using a previously described multiplex assay (50). Our group has recently used this assay to compare effector function responses in rabbits and rhesus macaques immunized with the same HIV vaccine (FIG. 13A-C).

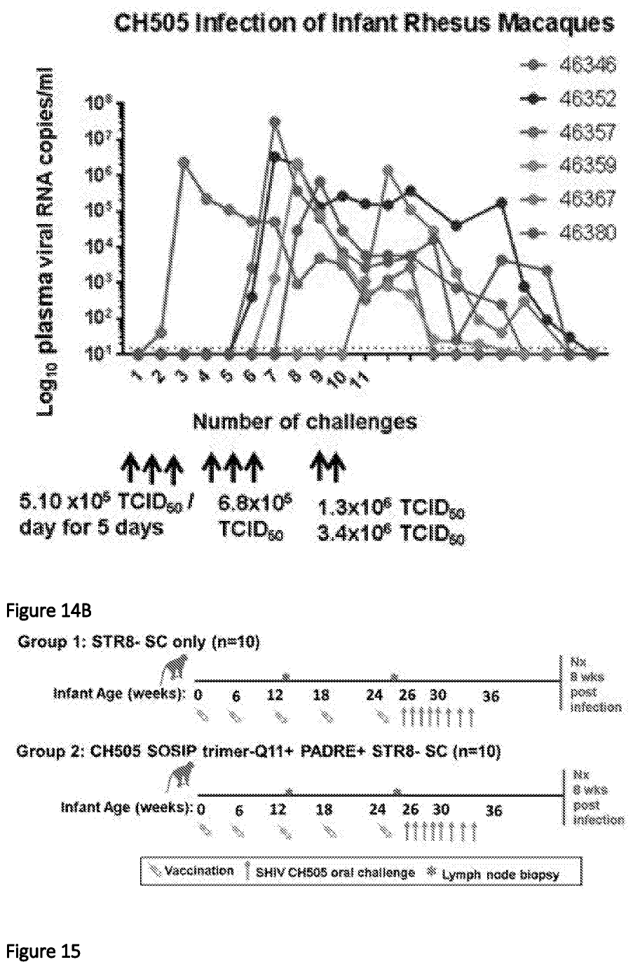

Immunogenicity study in infant rhesus macaques. We will conduct a pilot study in infant rhesus macaques to evaluate the immunogenicity of the best vaccine regimen from the rabbit immunization study. Selection of the P-Q11 trimer immunization regimen in rabbit studies will be based on primary criterion: P-Q11-CH505 trimer construct that induces the highest frequency/magnitude of tier 2 (autologous and heterologous neutralization) in rabbits, and secondary criteria 1-Highest magnitude of binding antibody responses, 2-Elicitation of non-neutralizing functional antibodies. In certain embodiments, a regimen will be defined as the P-Q11 CH505 trimer formulation that elicits the highest frequency/magnitude of tier 2 neutralization in rabbits. If there is no difference in the ability to elicit tier 2 neutralization between the regimens, additional criteria including magnitude of binding response and elicitation of non-neutralizing functional antibodies will also be taken into account. Six dam-reared infant macaques will be immunized at week 0, 6, 12, 18 and 24 (FIG. 14). Half of the macaques will receive the optimal regimen selected from the rabbit studies and the other half will receive a P-Q11 CH505 trimer construct containing a lower (3 microM) dose of PADRE as this dose was previously found to be optimal in primates (humans) (51). Blood will be collected prior to immunization and 2 weeks after each immunization and lymph nodes will be collected one week after the third and the final immunization. Animals will be necropsied 4 weeks after the last immunization.

Measurement of binding and functional antibody responses in infant rhesus macaques. We will assess the ability of the nanofiber vaccine to induce HIV Env specific binding antibodies by capture ELISA using an HRP conjugated anti-monkey secondary antibody. A polyclonal preparation of IgG purified from HIV vaccinated rhesus monkeys (RIVIG) will be used as standard. We will also map the epitope specificity and define the breadth of the vaccine-elicited antibodies by BAMA. In addition, we assess the binding of the vaccine-elicited antibodies to FcRs and measure non-neutralizing antibodies responses including binding to HIV infected cells and ADCC. Finally, to assess the induction of bnAb B cell lineage, we will measure plasma neutralization against autologous and heterologous tier 2 viruses after completion of the vaccine regimen and map the specificity of the detected neutralizing antibodies. We will also evaluate the ability of plasma from vaccinated animals to block the binding of bnAbs to the CH505 trimer by ELISA and measure HIV Env-specific B cells by flow cytometry.

Measurement of vaccine-elicited T follicular helper (Tfh) cells. As T cell help is critical for the induction of durable HIV-specific antibody responses, and for the affinity maturation required for neutralization breadth development, we will assess the ability of the CH505-Q11 PADRE vaccine to elicit robust T cell responses. The frequency of Tfh cells will be measured in the lymph nodes of the vaccinated infant rhesus macaque one week after the third and fifth immunization. Env-specific T follicular helper (Tfh) cell responses will be assessed using the Activation-Induced Marker (AIM) assay, in which antigen-specific Tfh cells upregulate surface markers, including OX40, CD25, and CD137 following incubation with antigen proteins and/or peptide pools (52). Additionally, we will characterize the phenotype of Tfh cells by flow cytometry using specific markers that indicate their underlying function. For example FoxP3 expression identifies Tfh, the regulatory subset of Tfh cells (53), whereas CXCR3⁺ CCR6⁻ Tfh cells exhibit a Th1 phenotype, CXCR3⁻ CCR6⁺ Tfh exhibit a Th17 phenotype, and CXCR3⁻ CCR6⁻ Tfh cells exhibit a Th2 phenotype (54). We will also measure vaccine-induced CD4+ and CD8+ T cell responses in peripheral blood and lymphoid tissues by intracellular cytokine staining.

Statistical analysis and power calculation. The statistical analysis will be done by the DHVI biostatistical unit. Our primary outcome in the rabbit study will be to compare vaccine-elicited immune responses between each of the P-Q11 CH505 trimer constructs and CH505 trimer alone. Based on previous data, with a sample size of 8 subjects and a standard deviation of log 2 titer of 1.3, a one tailed Wilcoxon test will provide 83.8% power to detect a 4.5-fold difference in autologous neutralization titers between the groups. The type-I error rate used is 0.0167, which includes a multiplicity adjustment of the nominal value of a 0.05 to allow for three multiple simultaneous hypothesis testing. Wilcoxon rank tests will be used to compare the three vaccine groups with the control group using Benjamini-Hochberg procedure to control the false discover rate. The pilot study in infant rhesus macaques is descriptive and is not powered for statistical analysis. Both female and male rabbits and rhesus macaque infants will be included in the immunogenicity studies. Rabbit studies will be performed with 4 animals per group per experiment, and then repeated once to make groups of 8, to avoid batch effects and assess repeatability. All assays will be conducted in duplicate, and generated data will be quality controlled using assay-specific criteria developed within each laboratory by lab staff that are blinded to the vaccination groups. Assays that fail pre-established QC will be repeated, and QC summaries will be generated to include data about potential repeats and the reason for repeat. Only data that pass QC will be reported. All materials will come from primary vendors or tested prior to use.

We expect that the immunogenicity of CH505 SOSIP-Q11 will superior to that of the CH505 SOSIP alone in rabbits and that we will be able to select a PADRE concentration for maximal elicitation of robust binding antibody responses and tier 2 neutralization. We also expect that P-Q11 CH 505 trimer will be immunogenic in infant rhesus macaques. These were defined taking into account previous results from immunization of adult rabbits with the stabilized CH505 SOSIP trimer (14).

The described assays are used in our laboratories and we do not anticipate difficulties in performing the proposed work. We propose to immunize the animals with five vaccine doses as rabbits demonstrated tier 2 neutralization after 2 to 4 immunizations in previous studies (14). If we do not achieve tier 2 neutralization titers, we will consider adding an additional boost. Finally, our study is limited by the lack of available reagents to define Tfh cells in rabbits. Thus, we will only be able to measure Tfh responses in non-human primates, but we will consider measuring T cell responses in rabbits by ELISPOT.

In certain aspects, the invention provides methods to determine the ability of the P-Q11CH505 trimer vaccine to protect against low dose oral SHIV challenge in an infant nonhuman primate model of late postnatal transmission via breastfeeding.

The ultimate approach to define if a vaccine strategy is potentially protective is to do a challenge study. Because our primary goal is to protect pediatric populations against breast milk transmission shortly after birth, but also elicit immunity that could be protective against sexual transmission at sexual debut, we propose to use an infant rhesus macaque oral challenge model using the recently engineered autologous SHIV CH505 (55). Our group has extensive experience with infant oral SHIV challenge models including with SHIV CH505 ((56), and FIG. 14). We hypothesize that Infant rhesus monkeys immunized with the P-Q11CH505 trimer vaccine will be protected against infection following low dose SHIV oral challenge as compared to unvaccinated animals.

Development of the SHIV CH505 infant macaque oral challenge model. In order to model breast milk transmission, six infant rhesus macaques were challenged orally with SHIV CH505, beginning at 4 weeks of age. Initially, infants were challenged three times per day with a bottle feeding for 5 days with SHIV.C.CH505 at a dose of 5.1×10⁵ TCID₅₀/day until infected. After three weeks, only one infant became infected, and the remaining five infants were subsequently challenged weekly under sedation at a dose of 6.8×10⁵ TCID₅₀ until infected. After three weekly challenges, only one infant remained uninfected and thus was subsequently challenged at increasing doses of 1.3×10⁶, followed by 3.4×10⁶ TCID₅₀ until infected (FIG. 14). This weekly oral challenge will be used to assess the efficacy of the P-Q11 CH505 trimer vaccine.

Infant Rhesus Macaque Challenge Study Design

Two groups of 10 infant rhesus macaques will be utilized in this study with equal numbers of male and female animals included in each group. See FIG. 15. The first group of animals will be immunized with 5 doses of the optimal P-Q11 CH505 trimer construct with STR8-SC as adjuvant. The vaccine dose of 15 μg of HIV Env protein will be administered intramuscularly in the quadriceps muscle every 6 weeks. The second group of monkeys will only receive adjuvant at the same time points as the vaccinated animals. Blood will be collected before immunization and 2 weeks after each immunization. A draining inguinal lymph node biopsy will be performed one week after the third and fifth immunization. Beginning two weeks after the final vaccine dose, the animals will be challenged weekly with a dose of 6.8×10⁵ TCID₅₀ of SHIV CH505. For SHIV CH505 see Bar K J, Coronado E, Hensley-McBain T, O'Connor M A, Osborn J M, Miller C, Gott T M, Wangari S, Iwayama N, Ahrens C Y, Smedley J, Moats C, Lynch R M, Haddad E K, Haigwood N L, Fuller D H, Shaw G M, Klatt N R, Manuzak J A. 2019. Simian-human immunodeficiency virus SHIV.CH505 infection of rhesus macaques results in persistent viral replication and induces intestinal immunopathology. J Virol 93:e00372-19. Starting at week 27, plasma samples will be tested weekly for viral RNA by RT PCR by Leidos Biomedical Research (J. Lifson) to identify animals who became infected. Animals with two consequences positive PCR results will be considered systemically infected with SHIV and will no longer be exposed to the virus. Animals with undetectable viral RNA (detection limit 15 copies/ml) will be rechallenged up to 15 times. The RMs will be followed for 8 weeks after either confirmed infection or the last challenge dose, then necropsied. Plasma viral load will be measured bi-weekly in infected animals until necropsy.

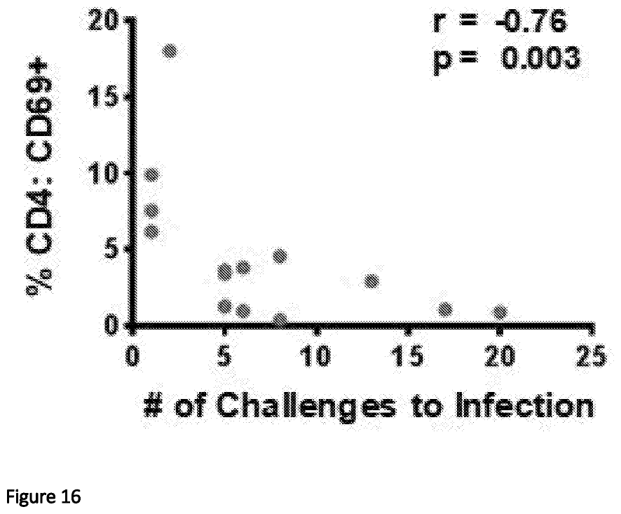

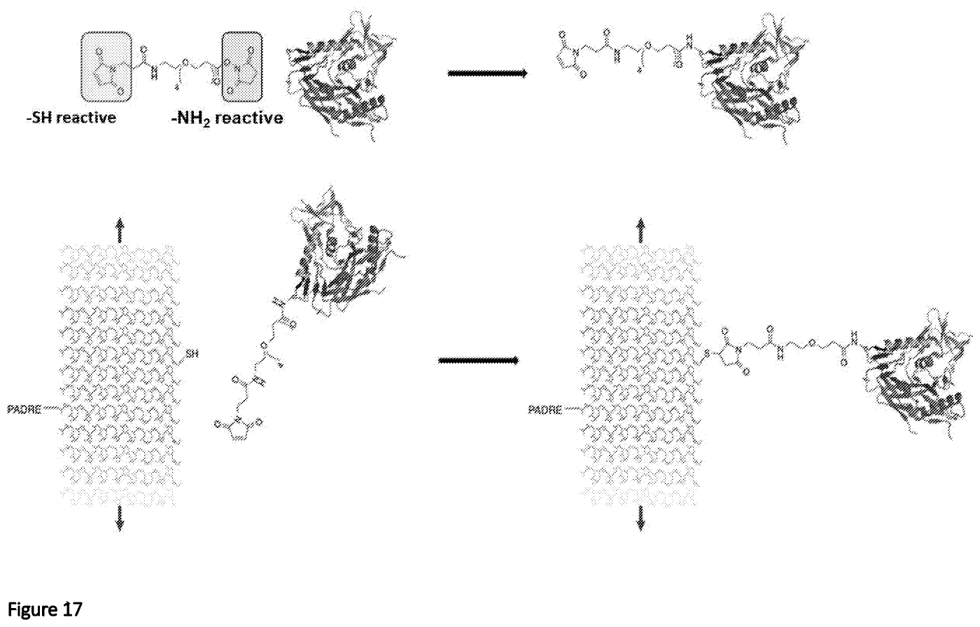

Measurement of immune responses pre- and post-virus exposure. Binding and functional antibody responses will be measured in the vaccinated animals at time points prior to virus exposure (week 2, 8, 14, 20 and 26) as described herein. In addition, we will assess the same antibody responses time points after challenge in vaccinated and controls animals who became infected as well as in protected vaccinated animals. Similarly, antigen-specific CD4+ and CD8+ T cell responses will be measured in peripheral blood and lymph nodes of vaccinated animals prior to virus exposure. We will also measure antigen-specific Tfh cells in lymph nodes as described in section C.2.3.2.2. In addition, we will define the activation phenotype of total CD4+ T cell in vaccinated and control animals at different time points prior to and following virus exposure as we previously observed an association between levels of activated CD4+ T cells prior to challenge and number of challenges required to infect infant rhesus macaques following oral SHIV exposure (56), (FIG. 16).

Statistical analysis and power calculation. Our primary outcome in this aim will be to assess the efficacy of P-Q11 CH505 trimer vaccine in infant macaques. We calculated the power for a Kaplan-Meier logrank test difference between the control and vaccine group under the following assumptions: the number of challenges to infection was assumed to be between 3-5 for the control group and between 6-15 for the vaccine group, at one side α=0.05 and N=10 per group. There is 80.8% power to detect a logrank difference when the median number of challenges to get infection in the control group is 3 and that in the treatment group is 11. Kaplan Meyer curves will be used to compare SHIV acquisition between the groups. We will use log rank test to compare the vaccine effect on number of challenges to infection at significance level of 0.05. In addition, we will do exploratory analysis using Benjamini-Hochberg procedure controlling the false discover rate. Moreover, we will fit a cox model using the proportion of activated CD4+ T cells as a predictor to predict the number of challenges to infection, and as a covariate to compare the vaccine effect.

Expected outcomes and potential future studies. If our hypothesis is correct, the number of vaccinated infant macaques who become infected will be significantly lower than the number of uninfected infant macaques who acquire infection. If it is the case, we will conduct an immune correlate analysis to identify immune factors that contributed to vaccine protection. In addition, if our strategy is able to induce tier 2 neutralizing antibodies, we will considered evaluating the B cell repertoire of the vaccinated animals to understand how neutralization breadth developed (57).

SHIV CH505 was selected as the challenge virus because 1) in this proof of concept study, we want to test the vaccine in an autologous virus challenge model. If successful, the vaccine strategy could subsequently be tested in a heterologous SHIV challenge model; and 2) we have previous experience working with this virus. Nevertheless, it is worth noting that while SHIV CH505 challenge results in a high peak virus load, some animals are able to control the virus spontaneously (see FIG. 14). Thus, we will not be able to determine if the vaccine-elicited immune responses contribute to virus control in vaccinated animals who become infected. Thus, our assessment of vaccine-induced protection will focus on prevention of virus acquisition and on the number of challenges required for infection.

### REFERENCES FOR EXAMPLE 1

1. World Health Organization. Global health sector strategy on HIV/AIDS 2000-2015: focus on innovations in Africa: progress report. Geneva, Switzerland: World Health Organization; 2015. Available from: apps.who.int/iris/bitstream/10665/198065/1/9789241509824_eng.pdf?ua=1.
2. Kesho Bora Study G, de Vincenzi I. Triple antiretroviral compared with zidovudine and single-dose nevirapine prophylaxis during pregnancy and breastfeeding for prevention of mother-to-child transmission of HIV-1 (Kesho Bora study): a randomised controlled trial. Lancet Infect Dis. 2011; 11(3):171-80.
3. Shapiro R L, Hughes M D, Ogwu A, Kitch D, Lockman S, Moffat C, et al. Antiretroviral regimens in pregnancy and breast-feeding in Botswana. N Engl J Med. 2010; 362(24):2282-94.
4. Lehman D A, Chung M H, John-Stewart G C, Richardson B A, Kiarie J, Kinuthia J, et al. HIV-1 persists in breast milk cells despite antiretroviral treatment to prevent mother-to-child transmission. AIDS. 2008; 22(12):1475-85.
5. Slyker J A, Chung M H, Lehman D A, Kiarie J, Kinuthia J, Holte S, et al. Incidence and correlates of HIV-1 RNA detection in the breast milk of women receiving HAART for the prevention of HIV-1 transmission. PLoS One. 2012; 7(1):e29777.
6. UNAIDS. HIV prevention among adolescent girls and young women. 2016.
7. PrabhuDas M, Adkins B, Gans H, King C, Levy O, Ramilo O, et al. Challenges in infant immunity: implications for responses to infection and vaccines. Nat Immunol. 2011; 12(3):189-94.
8. McGuire E P, Fong Y, Toote C, Cunningham C K, McFarland E J, Borkowsky W, et al. HIV-Exposed Infants Vaccinated with an MF59/Recombinant gp120 Vaccine Have Higher-Magnitude Anti-V1V2 IgG Responses than Adults Immunized with the Same Vaccine. J Virol. 2018; 92(1).
9. Dowling D J, Levy O. Ontogeny of early life immunity. Trends Immunol. 2014; 35(7):299-310.
10. Gray E S, Madiga M C, Hermanus T, Moore P L, Wibmer C K, Tumba N L, et al. The neutralization breadth of HIV-1 develops incrementally over four years and is associated with CD4+ T cell decline and high viral load during acute infection. J Virol. 2011; 85(10):4828-40.
11. Hraber P, Seaman M S, Bailer R T, Mascola J R, Montefiori D C, Korber B T. Prevalence of broadly neutralizing antibody responses during chronic HIV-1 infection. AIDS. 2014; 28(2):163-9.
12. Mikell I, Sather D N, Kalams S A, Altfeld M, Alter G, Stamatatos L. Characteristics of the earliest cross-neutralizing antibody response to HIV-1. PLoS Pathog. 2011; 7(1):e1001251.
13. Sanders R W, Moore J P. Native-like Env trimers as a platform for HIV-1 vaccine design. Immunol Rev. 2017; 275(1):161-82.
14. Saunders K O, Verkoczy L K, Jiang C, Zhang J, Parks R, Chen H, et al. Vaccine Induction of Heterologous Tier 2 HIV-1 Neutralizing Antibodies in Animal Models. Cell Rep. 2017; 21(13):3681-90.
15. Goo L, Chohan V, Nduati R, Overbaugh J. Early development of broadly neutralizing antibodies in HIV-1-infected infants. Nat Med. 2014; 20(6):655-8.
16. Muenchhoff M, Adland E, Karimanzira O, Crowther C, Pace M, Csala A, et al. Nonprogressing HIV-infected children share fundamental immunological features of nonpathogenic SIV infection. Sci Transl Med. 2016; 8(358):358ra125.
17. Ditse Z, Muenchhoff M, Adland E, Jooste P, Goulder P, Moore P L, et al. Hiv-1 Subtype C Infected Children with Exceptional Neutralization Breadth Exhibit Polyclonal Responses Targeting Known Epitopes. J Virol. 2018.
18. Walker L M, Simek M D, Priddy F, Gach J S, Wagner D, Zwick M B, et al. A limited number of antibody specificities mediate broad and potent serum neutralization in selected HIV-1 infected individuals. PLoS Pathog. 2010; 6(8):e1001028.
19. Simonich C A, Williams K L, Verkerke H P, Williams J A, Nduati R, Lee K K, et al. HIV-1 Neutralizing Antibodies with Limited Hypermutation from an Infant. Cell. 2016; 166(1):77-87.
20. Rudra J S, Sun T, Bird K C, Daniels M D, Gasiorowski J Z, Chong A S, et al. Modulating adaptive immune responses to peptide self-assemblies. ACS Nano. 2012; 6(2):1557-64.
21. Rudra J S, Tian Y F, Jung J P, Collier J H. A self-assembling peptide acting as an immune adjuvant. Proc Natl Acad Sci USA. 2010; 107(2):622-7.
22. Pompano R R, Chen J, Verbus E A, Han H, Fridman A, McNeely T, et al. Titrating T-cell epitopes within self-assembled vaccines optimizes CD4+ helper T cell and antibody outputs. Adv Healthc Mater. 2014; 3(11):1898-908.
23. Mora-Solano C, Wen Y, Han H, Chen J, Chong A S, Miller M L, et al. Active immunotherapy for TNF-mediated inflammation using self-assembled peptide nanofibers. Biomaterials. 2017; 149:1-11.
24. Chen J, Pompano R R, Santiago F W, Maillat L, Sciammas R, Sun T, et al. The use of self-adjuvanting nanofiber vaccines to elicit high-affinity B cell responses to peptide antigens without inflammation. Biomaterials. 2013; 34(34):8776-85.
25. Wen Y, Waltman A, Han H, Collier J H. Switching the Immunogenicity of Peptide Assemblies Using Surface Properties. ACS Nano. 2016.
26. Si Y, Wen Y, Kelly S H, Chong A S, Collier J H. Intranasal delivery of adjuvant-free peptide nanofibers elicits resident CD8(+) T cell responses. J Control Release. 2018; 282:120-30.
27. Jung J P M J, Collier J H. Multifactorial optimization of endothelial cell growth using modular synthetic extracellular matrices. Integr Biol (Camb) 2011 March; 3(3):185-96 doi: 101039/c0ib00112k Epub 2011 Jan. 19, 2011.
28. Jung J P N A, Fox E K, Rudra J S, Devgun J M, Collier J H. Co-assembling peptides as defined matrices for endothelial cells. Biomaterials 2009; April; 30(12):2400-10. doi: 10.1016/j.biomaterials.2009.01.033. Epub 2009 Feb. 8.
29. Hudalla G A, Sun T, Gasiorowski J Z, Han H, Tian Y F, Chong A S, et al. Gradated assembly of multiple proteins into supramolecular nanomaterials. Nat Mater. 2014; 13(8):829-36.
30. Hudalla G A M J, Tian Y F, Rudra J S, Chong A S, Sun T, Mrksich M, Collier J H. A self-adjuvanting supramolecular vaccine carrying a folded protein antigen. Adv Healthc Mater. 2013; 2(8:1114-9. doi: 10.002/adhm.201200435. Epub 2013 Feb. 25.
31. Mora-Solano C W Y, Han H, Chen J, Chong A S, Miller M L, Pompano R R, Collier J H. Active immunotherapy for TNF-mediated inflammation using self-assembled peptide nanofibers. Biomaterials 149:1-11 doi: 101016/jbiomaterials201709031 Epub 2017 Sep. 26 2017.
32. Gao F, Bonsignori M, Liao H X, Kumar A, Xia S M, Lu X, et al. Cooperation of B cell lineages in induction of HIV-1-broadly neutralizing antibodies. Cell. 2014; 158(3):481-91.
33. Zhou T, Doria-Rose N A, Cheng C, Stewart-Jones G B E, Chuang G Y, Chambers M, et al. Quantification of the Impact of the HIV-1-Glycan Shield on Antibody Elicitation. Cell Rep. 2017; 19(4):719-32.
34. de Taeye S W, Ozorowski G, Torrents de la Pena A, Guttman M, Julien J P, van den Kerkhof T L, et al. Immunogenicity of Stabilized HIV-1 Envelope Trimers with Reduced Exposure of Non-neutralizing Epitopes. Cell. 2015; 163(7):1702-15.
35. Batista F D, Neuberger M S. B cells extract and present immobilized antigen: implications for affinity discrimination. EMBO J. 2000; 19(4):513-20.
36. Ingale J, Stano A, Guenaga J, Sharma S K, Nemazee D, Zwick M B, et al. High-Density Array of Well-Ordered HIV-1 Spikes on Synthetic Liposomal Nanoparticles Efficiently Activate B Cells. Cell Rep. 2016; 15(9):1986-99.
37. Martinez-Murillo P, Tran K, Guenaga J, Lindgren G, Adori M, Feng Y, et al. Particulate Array of Well-Ordered HIV Clade C Env Trimers Elicits Neutralizing Antibodies that Display a Unique V2 Cap Approach. Immunity. 2017; 46(5):804-17 e7.
38. Solano C M, Wen Y, Han H, Collier J H. Practical Considerations in the Design and Use of Immunologically Active Fibrillar Peptide Assemblies. Methods Mol Biol. 2018; 1777:233-48.
39. Moody M A, Santra S, Vandergrift N A, Sutherland L L, Gurley T C, Drinker M S, et al. Toll-like receptor 7/8 (TLR7/8) and TLR9 agonists cooperate to enhance HIV-1 envelope antibody responses in rhesus macaques. J Virol. 2014; 88(6):3329-39.
40. Phillips B, Van Rompay, K., Rodriguez-Nieves, J., Lorin, C., Koutsoukos, M., Tomai, M., r Fox, C., Eudailey, J., Dennis, M., Alam, S M., Hudgens, M., Fouda, G., Pollara, J., Moody, M A., Shen, X., Ferrari, G., Permar, S., and K. De Paris. Adjuvant-dependent enhancement of HIV Env-specific antibody responses in infant rhesus macaques. in press, JVI01051-18.
41. Lai C Y, Liu Y L, Yu G Y, Maa M C, Leu T H, Xu C, et al. TLR7/8 agonists activate a mild immune response in rabbits through TLR8 but not TLR7. Vaccine. 2014; 32(43):5593-9.
42. Fouda G G, Cunningham C K, McFarland E J, Borkowsky W, Muresan P, Pollara J, et al. Infant HIV type 1 gp120 vaccination elicits robust and durable anti-V1V2 immunoglobulin G responses and only rare envelope-specific immunoglobulin A responses. J Infect Dis. 2015; 211(4):508-17.
43. Phillips B, Fouda G G, Eudailey J, Pollara J, Curtis A D, 2nd, Kunz E, et al. Impact of Poxvirus Vector Priming, Protein Coadministration, and Vaccine Intervals on HIV gp120 Vaccine-Elicited Antibody Magnitude and Function in Infant Macaques. Clin Vaccine Immunol. 2017; 24(10).
44. Sanders R W, Derking R, Cupo A, Julien J P, Yasmeen A, de Val N, et al. A next-generation cleaved, soluble HIV-1 Env trimer, BG505 SOSIP.664 gp140, expresses multiple epitopes for broadly neutralizing but not non-neutralizing antibodies.

PLoS Pathog. 2013; 9(9):e1003618.
45. Chung A W, Ghebremichael M, Robinson H, Brown E, Choi I, Lane S, et al. Polyfunctional Fc-effector profiles mediated by IgG subclass selection distinguish RV144 and VAX003 vaccines. Sci Transl Med. 2014; 6(228):228ra38.
46. Haynes B F, Gilbert P B, McElrath M J, Zolla-Pazner S, Tomaras G D, Alam S M, et al. Immune-correlates analysis of an HIV-1 vaccine efficacy trial. N Engl J Med. 2012; 366(14):1275-86.
47. Bradley T, Pollara J, Santra S, Vandergrift N, Pittala S, Bailey-Kellogg C, et al. Pentavalent HIV-1 vaccine protects against simian-human immunodeficiency virus challenge. Nat Commun. 2017; 8:15711.
48. Gordon S N, Liyanage N P, Doster M N, Vaccari M, Vargas-Inchaustegui D A, Pegu P, et al. Boosting of ALVAC-SIV Vaccine-Primed Macaques with the CD4-SIVgp120 Fusion Protein Elicits Antibodies to V2 Associated with a Decreased Risk of SIVmac251 Acquisition. J Immunol. 2016; 197(7):2726-37.
49. Barouch D H, Alter G, Broge T, Linde C, Ackerman M E, Brown E P, et al. Protective efficacy of adenovirus/protein vaccines against SIV challenges in rhesus monkeys. Science. 2015; 349(6245):320-4.
50. Brown E P, Dowell K G, Boesch A W, Normandin E, Mahan A E, Chu T, et al. Multiplexed Fc array for evaluation of antigen-specific antibody effector profiles. J Immunol Methods. 2017; 443:33-44.
51. Rojas J M, Knight K, Wang L, Clark R E. Clinical evaluation of BCR-ABL peptide immunisation in chronic myeloid leukaemia: results of the EPIC study. Leukemia. 2007; 21(11):2287-95.
52. Reiss S, Baxter A E, Cirelli K M, Dan J M, Morou A, Daigneault A, et al. Comparative analysis of activation induced marker (AIM) assays for sensitive identification of antigen-specific CD4 T cells. PLoS One. 2017; 12(10):e0186998.
53. Chung Y, Tanaka S, Chu F, Nurieva R I, Martinez G J, Rawal S, et al. Follicular regulatory T cells expressing Foxp3 and Bcl-6 suppress germinal center reactions. Nat Med. 2011; 17(8):983-8.
54. Morita R, Schmitt N, Bentebibel S E, Ranganathan R, Bourdery L, Zurawski G, et al. Human blood CXCR5(+)CD4(+) T cells are counterparts of T follicular cells and contain specific subsets that differentially support antibody secretion. Immunity. 2011; 34(1):108-21.
55. Li H, Wang S, Kong R, Ding W, Lee F H, Parker Z, et al. Envelope residue 375 substitutions in simian-human immunodeficiency viruses enhance CD4 binding and replication in rhesus macaques. Proc Natl Acad Sci USA. 2016; 113(24):E3413-22.
56. Eudailey J A, Dennis M L, Parker M E, Phillips B L, Huffman T N, Bay C P, et al. Maternal HIV-1 Env Vaccination for Systemic and Breast Milk Immunity To Prevent Oral SHIV Acquisition in Infant Macaques. mSphere. 2018; 3(1).
57. Williams W B, Zhang J, Jiang C, Nicely N I, Fera D, Luo K, et al. Initiation of HIV neutralizing B cell lineages with sequential envelope immunizations. Nat Commun. 2017; 8(1):1732.

FIGS. 10D and 10-E, show improvement of poor B-cell responses in mice—where one embodiment of the inventive formulation is compared to antigen/adjuvant alone. In FIG. 10E, the additional improvement of B-cell responses via incorporation of T-cell epitopes is shown. These data show that nanofiber formulations of the invention increase poor B-cell responses raised by this the evaluated antigen.

Direct ELISA was adopted to probe the anti-1086c gp120 antibody response in C57BL/6 mice vaccinated with 2 doses of 15 μg Q11 nanofiber-conjugated 1086c gp120 or plain 1086c gp120 in the presence of 10% (v/v) STR8S-C(Slide13). The same method was used to test the antibody response elicited by 2 doses of 15 g 1086c gp120 with or without PADRE-Q11 incorporation and 2 doses of 50 g 1086c gp120 with PADRE-Q11 conjugation in mice (Slide 14). The total peptide concentration in each formulation is 2 mM (cQ11 plus Q11). In PADRE-containing formulations, there is 0.1 mM PADRE-Q11 included, with 1.9 mM cQ11+Q11.

Immunization schedule: C57BL/6 mice were subcutaneously immunized with 2 doses of 15 g 1086c gp120 (n=5) or 15 g 1086c gp120 conjugated to Q11 nanofibers (n=5) in the presence of 10% STR8S-C adjuvant in week0 and week11 (Slide13). Another cohort received 2 doses of 15 g 1086c gp120 with or without PADRE-Q11 included in the nanofibers (n=10 for each) or 2 doses of 50 g 1086c gp120 with PADRE-Q11 included (n=10) in week0 and week7.

The studies in FIGS. 10D and 10E are the same studies discussed in FIG. 10A-C of Ex 1. FIGS. 10D and 10E show additional time points as the animal studies progressed further.

### Example 2

Conjugation of Envelope to Nanofiber

Linkage of 1086.c gp120 proteins to Q11 nanofibers is mediated by a two-step reaction in which proteins are modified with a heterobifunctional cross linker with amine- and thiol-reactive groups and subsequently linked to cysteine decorated nanofibers. Sulfosuccinimidyl 4-(N-maleimidomethyl)cyclohexane-1-carboxylate (Sulfo-SMCC) and succinimidyl-[(N-maleimidopropionamido)-octaethyleneglycol] ester (SM-PEG8) were both used to successfully link nanofibers to proteins (see structures below). When developing the synthesis, both cross linkers were tested in parallel and the solvent composition, reaction temperature and stoichiometry were varied to optimize reaction efficiency and binding of gp120-specific antibodies to conjugated products. Additionally, processing of the modified proteins and preparation of nanofibers was systematically varied to increase protein loading on nanofibers and preserve their structure. To achieve this, the reduction of thiol groups on nanofibers was tested using soluble and particle-bound (tris(2-carboxyethyl)phosphine) (TCEP). Particle bound TCEP was found to be minimally damaging to proteins while soluble TCEP reduced antibody binding after synthesis. After modifying proteins with SMCC or SM-PEG8, dialysis in PBS at 4° C. was found to preserve protein structure most effectively, as measured by antibody binding. Finally, nanofiber morphology was preserved by decreasing centrifugation speed in the final step of product purification. Each of the aforementioned conditions was optimized by testing constructs with direct ELISA, SDS PAGE, and biolayer interferometry.

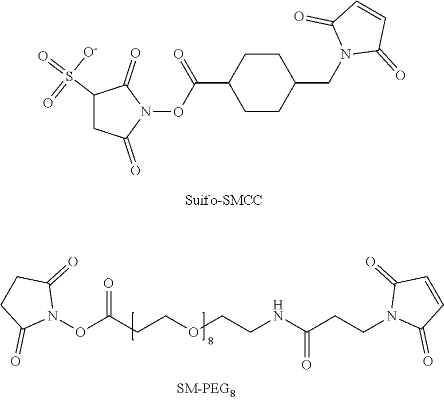

### Example 3

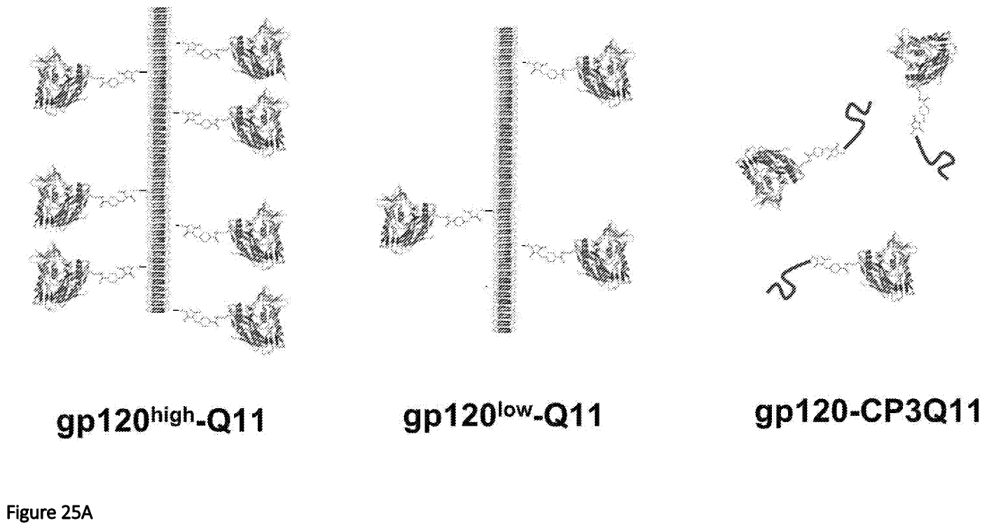

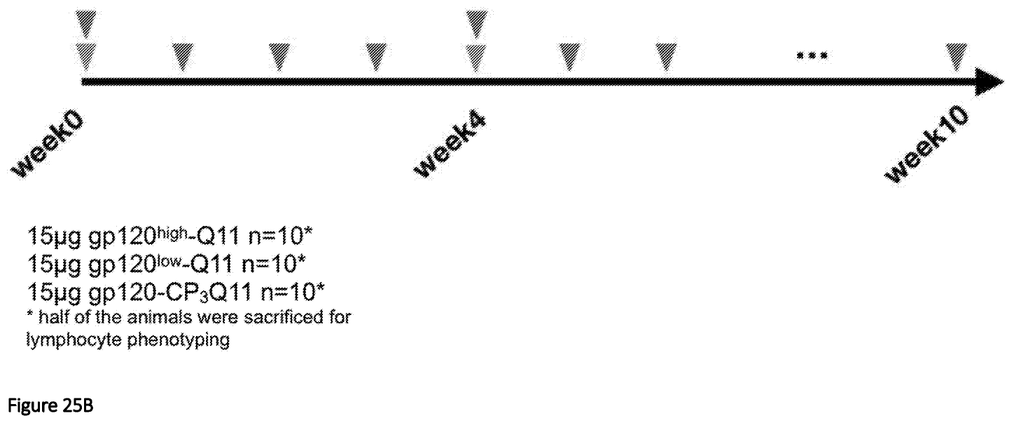

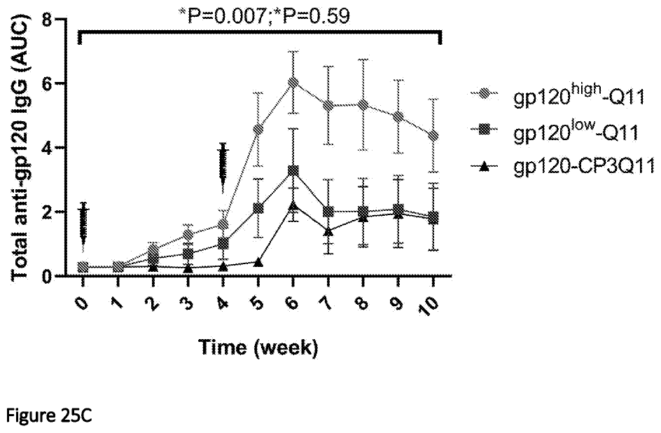

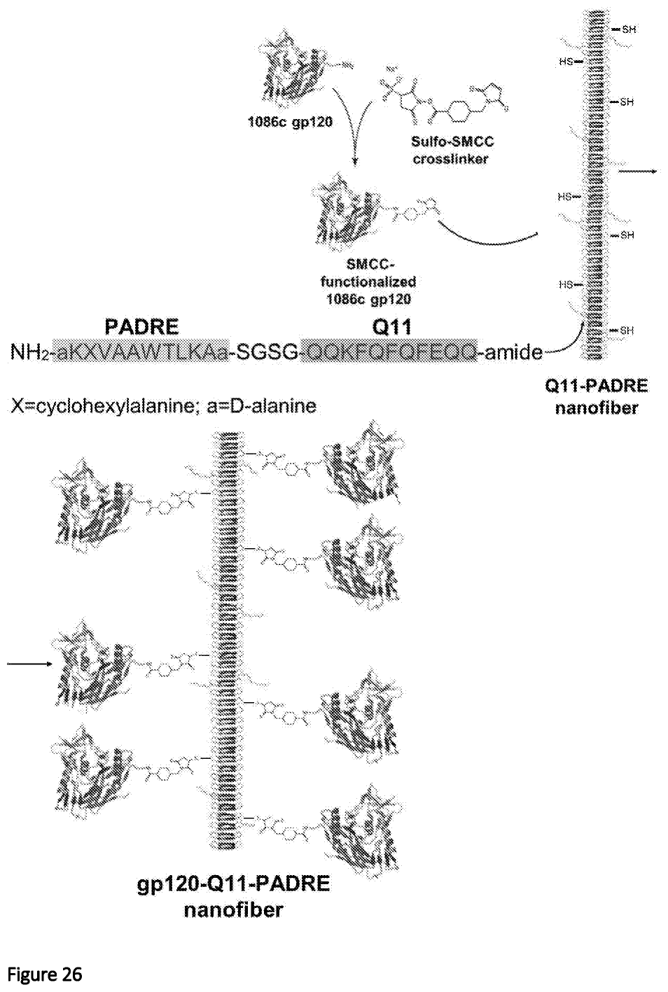

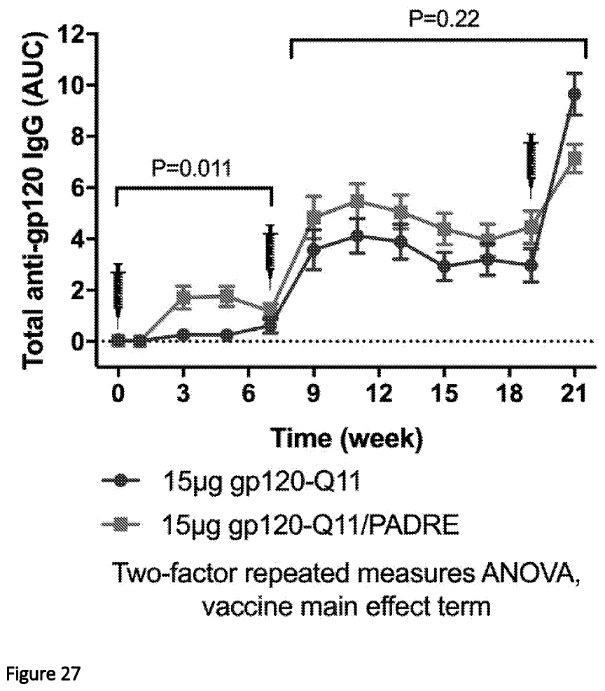

This example provides experiments on several aspects of the invention. The example shows that multivalency is important for the elevated antibody response induced by gp120-Q11 compared with different gp120 density. See e.g. FIG. 25A-25C.

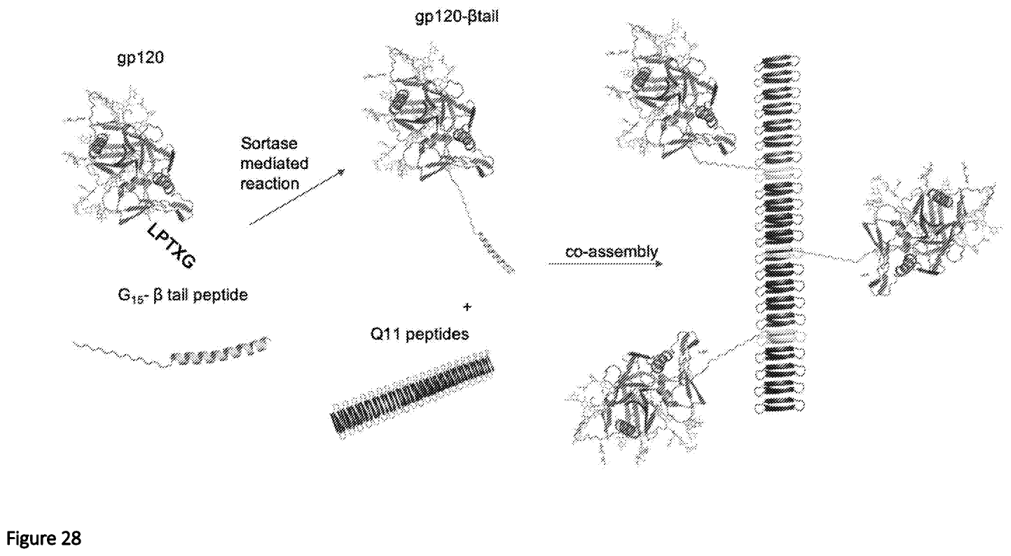

See FIGS. 28-32 and accompanying description. To improve the antigenicity of Q11-conjugated HIV vaccines, a site-specific sortase-mediated conjugation method was used to synthesize the nanofibers. Sortase A catalyzes a covalent conjugation between a LPXTG amino acid sequence and a second polyglycine peptide. This enzyme was used to link CH505 gp120 expressed with LPXTG tag to a β-tail peptide synthesized in tandem with a polyglycine tail. By co-assembling the β-tail-modified CH505 gp120 with Q11 peptide, nanofibers bearing CH505 gp120 were formed. FIG. 28.

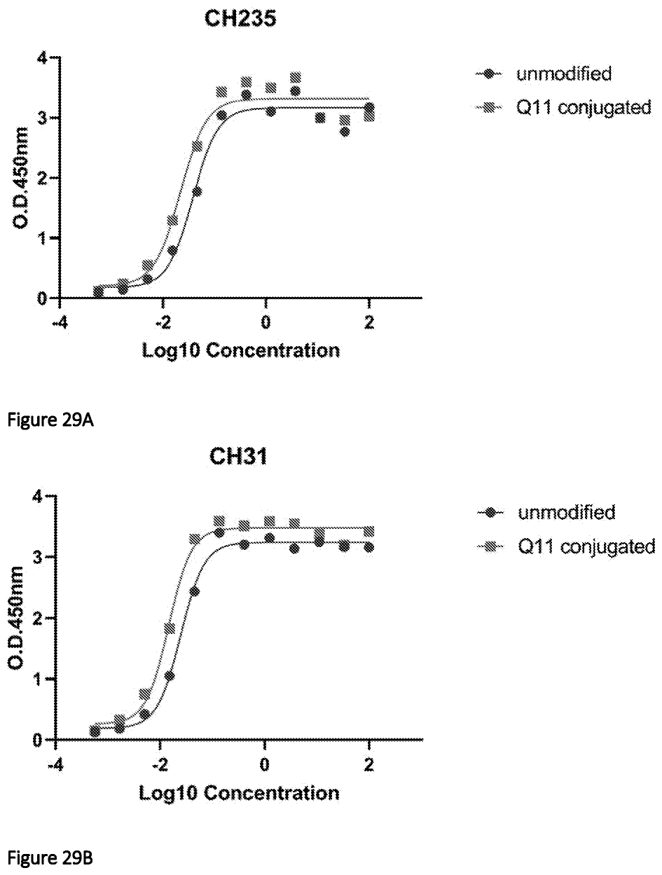

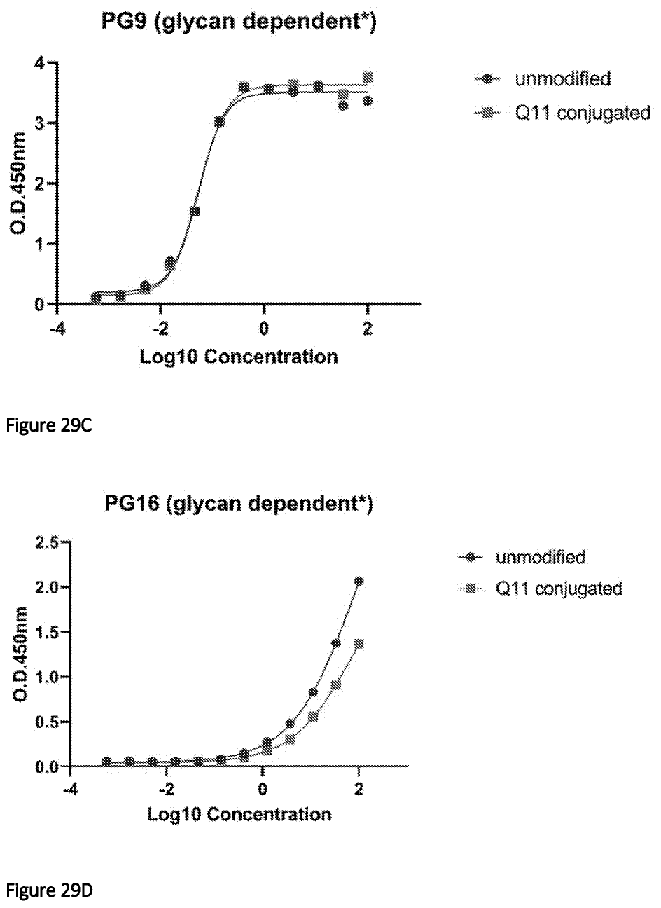

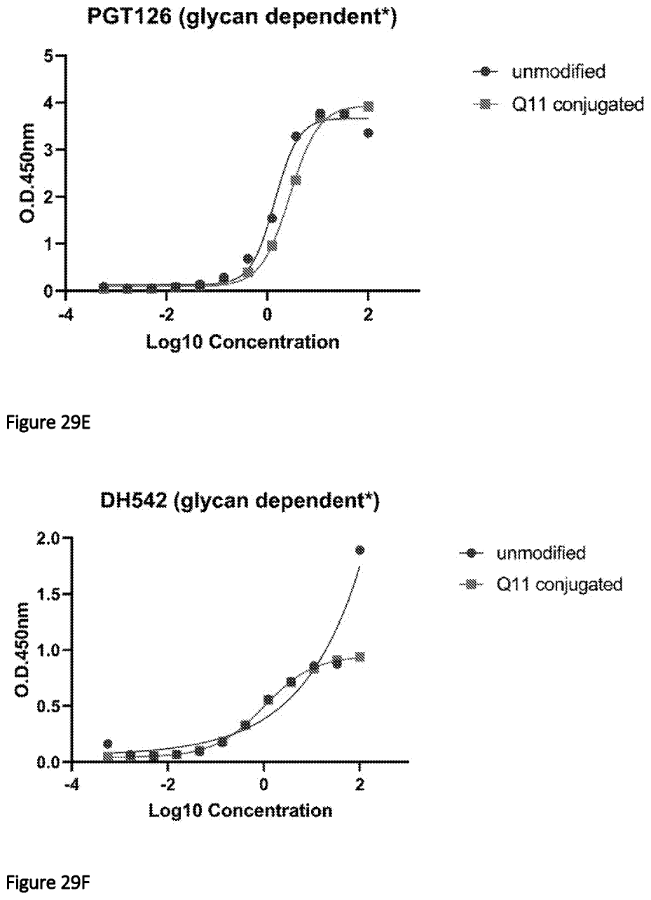

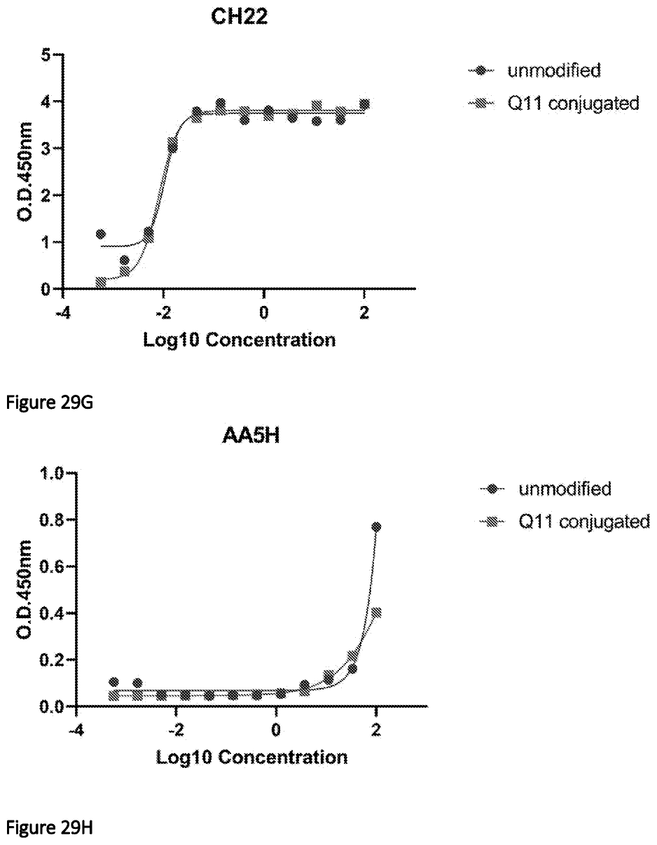

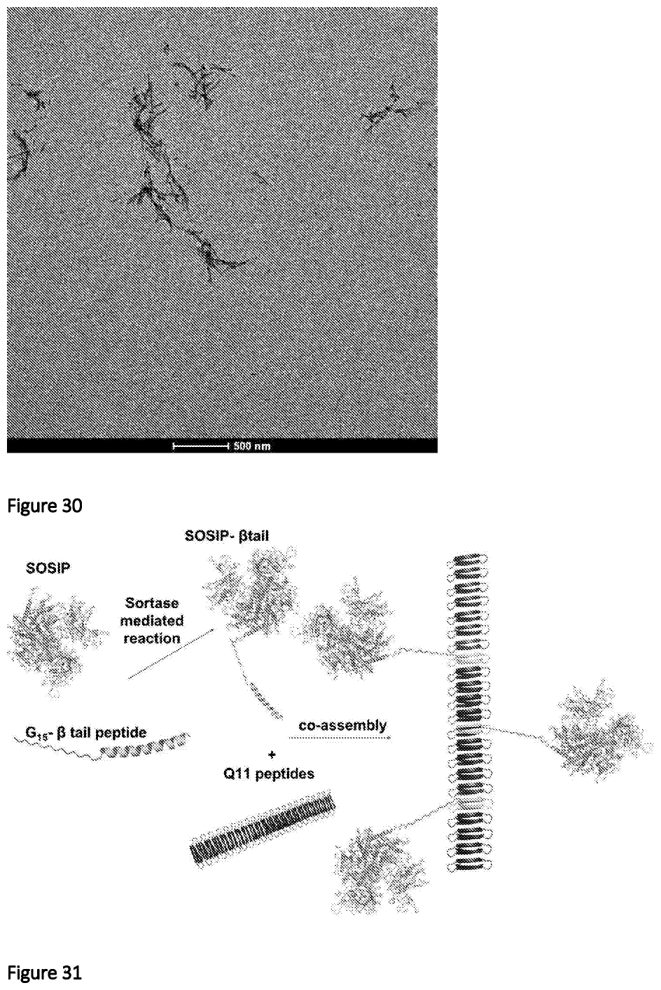

The antigenicity of the Q11-sortase conjugated gp120 was evaluated by ELISA by comparing the binding of a panel of mAbs to the unmodified gp120 and β-tail tagged gp120 incorporated into Q11 nanofibers. The binding of all the tested mAbs was comparable between the conjugated and unconjugated gp120 for all the HIV-specific mAbs including the CD4 binding site mAb Ch31 and CH235.12, the V2 glycan dependent mAbs PG9 and PG16, the V3 glycan dependent mAb PGT126, and the V3-specific mAb Ch22. Nanofiber morphology was assessed by TEM. FIGS. 29-30

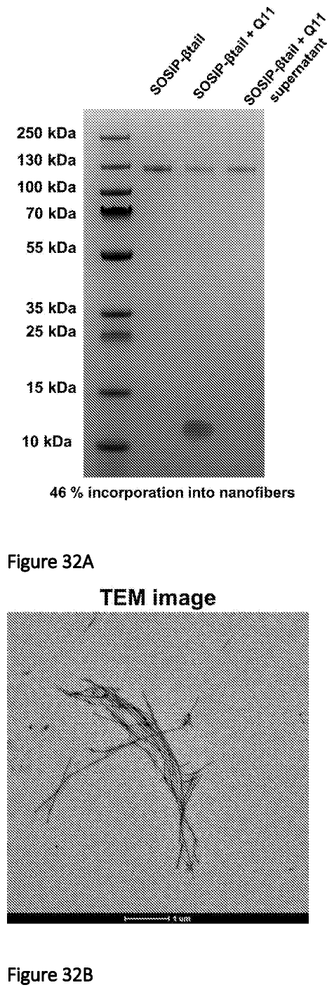

The sortase-based approach was also used to conjugate CH505 SOSIP trimers to Q11. In FIGS. 31-32, LPETG-tagged SOSIP trimer was conjugated to a synthesized polyglycine (G15)-modified β-tail peptide using a sortase enzyme. The enzyme was then removed, and the β-tail tagged SOSIP was mixed with fibrillizing Q11 peptides. After mixing these components, nanofibers were centrifuged and washed to remove any SOSIP that was not incorporated in nanofiber assemblies. ˜50% of the SOSIP trimer was incorporated in to nanofibers as measured by SDS PAGE.

The SOSIP HIV antigen that induced antibodies that neutralize HIV was conjugated to Q11 nanofiber. For one embodiment, see FIGS. 31-32. Further studies are needed to determine whether there is a more site-specific conjugation method to avoid disruption of trimer structure, and to optimize the use of sortase-mediated conjugation. The SOSIP nanofiber will be analyzed for antigenicity. A rabbit and or NHP study will address the immunogenicity of HIV envelope SOSIP trimer conjugated to nanofiber.

What is claimed is:

1. An immunogenic composition comprising a nanofiber complex composition, wherein the composition comprises a β-sheet nanofiber structure comprising a plurality of β-sheet peptides, and at least one compound linked to at least one of the β-sheet peptides, and wherein the compound is an HIV-1 envelope such as gp120, gp140, or a stabilized trimer.

2. The immunogenic composition of claim 1 wherein the at least one compound is linked to at least one of the β-sheet peptides via any suitable linker.

3. The composition of claim 1 or 2, wherein the compound is gp120 HIV-1 envelope 1086.C.

4. The composition of claim 1 or 2, wherein the compound is an HIV-1 envelope trimer.

5. The composition of any of the preceding claims, wherein the plurality of β-sheet peptides comprises a plurality of self-assembling peptides.

6. The composition of any of the preceding claims, wherein the β-sheet peptide is Q11.

7. The composition of any of the preceding claims, wherein the nanofiber complex composition further comprises a T-cell epitope peptide.

8. The composition of any of the preceding claims, wherein the nanofiber complex composition further comprises the PADRE peptide.

9. The composition of any one of the preceding claims further comprising an adjuvant.

10. A method of inducing an immune response in a subject, comprising administering to the subject any one of the compositions of claim 1-9 in an amount suitable to effect such induction.

11. The method of claim 10 further comprising administering an adjuvant.
